(19) United States
(12) Patent Application Publication
(10) Pub. No.: US 2023/0332228 A1
(43) Pub. Date: Oct. 19, 2023

(54) METHODS AND SYSTEMS FOR DETERMINING EFFECTS OF NUCLEIC ACID EDITING

(71) Applicant: BAR ILAN UNIVERSITY, Ramat Gan (IL)

(72) Inventors: Ayal HENDEL, Kiryat Ono (IL); Zohar YAKHINI, Ramat Hasharon (IL); Ido AMIT, Tel Aviv (IL)

(21) Appl. No.: 17/998,722

(22) PCT Filed: May 11, 2021
(86) PCT No.: PCT/IL2021/050539

§ 371 Date: Nov. 14, 2022

Related U.S. Application Data
(60) Provisional application No. 63/022,912, filed on May 11, 2020.

(51) Int. Cl. C12Q 1/6876; G16B 30/00; G16B 25/20; G16B 40/00

(52) U.S. Cl. CPC C12Q 1/6876; G16B 30/00; G16B 25/20; G16B 40/00; C12Q 2600/16; C12Q 2600/166

(57) ABSTRACT

Novel systems methods and kits are disclosed for determining effects of Nucleic Acid (NA) editing procedures. The technique (methods systems and kits) of the present invention are adapted for receiving sequencing data indicative of pluralities of reads, R_{Tx}={ri^{Tx}} and R_{Mc}={rj^{Mc}}, of the multiplexed amplifications of each of the edit and control NA collections, originating from a similar source of NAs whereby a certain NA editing procedure was applied to the edit NA collection; and processing the sequencing data, per each particular type of adverse effect of interest to determine a statistical model for classifying whether that type of adverse effect actually occurred due to the NA editing procedure. The technique is suitable for determining occurrences of INDEL types and/or TRANSLOCATION types/species of adverse effect. The technique further enables to statistically quantify the rates of occurrence types of adverse effects which are actually caused by the NA editing procedure, and also to provide statistical confidence intervals for these rates according to a desired statistical confidence level.

### TECHNOLOGICAL FIELD

The present invention is in the field of Nucleic Acid (NA) editing, such as gene editing, and more specifically relates to techniques for determining the effects of an NA editing procedure on Nucleic Acids (NA) acquired from a given source.

### BACKGROUND ART

References considered to be relevant as background to the presently disclosed subject matter are listed below:
1. Porteus, M. H. A New Class of Medicines through DNA Editing. N. Engl. J. Med. 380, 947-959(2019).
2. Tsai, S. Q. & Joung, J. K. Defining and improving the genome-wide specificities of CRISPR-Cas9 nucleases. Nat. Rev. Genet. 17, 300-312 (2016).
3. Bikard, D. et al. Programmable repression and activation of bacterial gene expression using an RNA-guided DNA binding protein Supplementary Materials. Nucleic Acids Res. 41, 7429-7437 (2013).
4. Zhang, Y., Malzahn, A. A., Sretenovic, S. & Qi, Y. The emerging and uncultivated potential of CRISPR technology in plant science. Nature Plants vol. 5 778-794 (2019).
5. Han, R. et al. Functional CRISPR screen identifies AP1-associated enhancer regulating FOXF1 to modulate oncogene-induced senescence. Genome Biol. 19, (2018).
6. Irion, U., Krauss, J. & Nusslein-Volhard, C. Precise and efficient genome editing in zebrafish using the CRISPR/Cas9 system. Dev. 141, 4827-4830 (2014).
7. Kim, D., Luk, K., Wolfe, S. A. & Kim, J. S. Evaluating and enhancing target specificity of gene-editing nucleases and deaminases. Annu. Rev. Biochem. 88, 191-220 (2019).
8. Tsai, S. Q. et al. GUIDE-seq enables genome-wide profiling of off-target cleavage by CRISPR-Cas nucleases. Nat. Biotechnol. 33, 187-198 (2015).
9. Tsai, S. Q. et al. CIRCLE-seq: A highly sensitive in vitro screen for genome-wide CRISPR-Cas9 nuclease off-targets. Nat. Methods 14, 607-614 (2017).
10. Cameron, P. et al. Mapping the genomic landscape of CRISPR-Cas9 cleavage. Nat. Methods 14, 600-606 (2017).
11. Wienert, B. et al. Unbiased detection of CRISPR off-targets in vivo using DISCOVER-Seq. Science (80-.). 364, 286-289 (2019).
12. Shapiro, J. et al. Increasing CRISPR efficiency and measuring its specificity in hematopoietic stem and progenitor cells using a clinically relevant system. Mol. Ther.—Methods Clin. Dev. (2020) https://doi.org/10.1016/j.omtm.2020.04.027.
13. Vakulskas, C. A. et al. A high-fidelity Cas9 mutant delivered as a ribonucleoprotein complex enables efficient gene editing in human hematopoietic stem and progenitor cells. Nat. Med. 24, 1216-1224 (2018).
14. Mitelman, F., Johansson, B. & Mertens, F. The impact of translocations and gene fusions on cancer causation. Nature Reviews Cancer vol. 7 233-245 (2007).
15. Wilch, E. S. & Morton, C. C. Historical and clinical perspectives on chromosomal translocations. in Advances in Experimental Medicine and Biology vol. 1044 1-14 (Springer New York LLC, 2018).
16. Roukos, V. & Misteli, T. The biogenesis of chromosome translocations. Nature Cell Biology vol. 16 293-300 (2014).
17. Zheng, Z. et al. Anchored multiplex PCR for targeted next-generation sequencing. Nat. Med. 20, 1479-1484 (2014).
18. Frock, R. L. et al. Genome-wide detection of DNA double-stranded breaks induced by engineered nucleases. Nat. Biotechnol. 33, 179-188 (2015).
19. Giannoukos, G. et al. UDiTaS™, A genome editing detection method for indels and genome rearrangements. BMC Genomics 19, (2018).
20. Huang, D. W. et al. The DAVID Gene Functional Classification Tool: A novel biological module-centric algorithm to functionally analyze large gene lists. Genome Biol. 8, R183 (2007).
21. Robinson, J. T. et al. Integrative genomics viewer. Nature Biotechnology vol. 29 24-26 (2011).
22. Eden, E., Navon, R., Steinfeld, I., Lipson, D. & Yakhini, Z. GOrilla: A tool for discovery and visualization of enriched GO terms in ranked gene lists. BMC Bioinformatics 10, 48 (2009).
23. Kent, W. J. et al. The Human Genome Browser at UCSC. Genome Res. 12, 996-1006 (2002).
24. Guell, M., Yang, L. & Church, G. M. Genome editing assessment using CRISPR Genome Analyzer (CRISPR-GA). Bioinformatics 30, 2968-2970 (2014).
25. Park, J., Lim, K., Kim, J.-S. & Bae, S. Cas-analyzer: an online tool for assessing genome editing results using NGS data. Bioinformatics 33, 286-288 (2016).
26. Lindsay, H. et al. CrispRVariants charts the mutation spectrum of genome engineering experiments. Nature Biotechnology vol. 34 701-702 (2016).
27. Wang, X. et al. CRISPR-DAV: CRISPR NGS data analysis and visualization pipeline. Bioinformatics 33, 3811-3812 (2017).
28. Boel, A. et al. BATCH-GE: Batch analysis of Next-Generation Sequencing data for genome editing assessment. Sci. Rep. 6, 1-10 (2016).
29. Connelly, J. P. & Pruett-Miller, S. M. CRIS.py: A Versatile and High-throughput Analysis Program for CRISPR-based Genome Editing. Sci. Rep. 9, 1-8 (2019).
30. Hardwick, S. A., Deveson, I. W. & Mercer, T. R. Reference standards for next-generation sequencing. Nature Reviews Genetics vol. 18 473-484 (2017).
31. Pinello, L. et al. Analyzing CRISPR genome-editing experiments with CRISPResso. Nature Biotechnology vol. 34 695-697 (2016).
32. Clement, K. et al. CRISPResso2 provides accurate and rapid genome editing sequence analysis. Nature Biotechnology vol. 37 224-226 (2019).
33. Labun, K. et al. Accurate analysis of genuine CRISPR editing events with ampliCan. Genome Res. 29, 843-847 (2019).
34. Schubert, M. et al. Evaluate CRISPR-Cas9 edits quickly and accurately with rhAmpSeq targeted sequencing. www.idtdna.com.
35. Peng, Q., Vijaya Satya, R., Lewis, M., Randad, P. & Wang, Y. Reducing amplification artifacts in high multiplex amplicon sequencing by using molecular barcodes. BMC Genomics 16, (2015).
36. Chen, S., Zhou, Y., Chen, Y. & Gu, J. fastp: an ultra-fast all-in-one FASTQ preprocessor. Bioinformatics 34, i884-i890 (2018).
37. Needleman, S. B. & Wunsch, C. D. A general method applicable to the search for similarities in the amino acid sequence of two proteins. J. Mol. Biol. 48, 443-453 (1970).
38. Benjamini, Y. & Hochberg, Y. Controlling the False Discovery Rate: A Practical and Powerful Approach to Multiple Testing. J. R. Stat. Soc. Ser. B 57, 289-300 (1995).
39. Dobosy, J. R. et al. RNase H-dependent PCR (rhPCR): Improved specificity and single nucleotide polymorphism detection using blocked cleavable primers. BMC Biotechnol. 11, 1-18 (2011).
40. Myles Hollander; Douglas A. Wolfe; Eric Chicken. Nonparametric Statistical Methods. in (2014).
41. Amit, I. et al. CRISPECTOR—Accurate estimation of genome editing translocation and off-target activity from comparative NGS data. GitHub repository. doi: 10.5281/zenodo.4561518 (2021).
42. PCT patent application publication No. WO2020/123906. Acknowledgement of the above references herein is not to be inferred as meaning that these are in any way relevant to the patentability of the presently disclosed subject matter.

### BACKGROUND

Nucleic Acid (NA) editing techniques present powerful tools which may be used inter-alia for curing genetic illnesses, genome editing in mammalians, for enhancement as well as for treatment, in crop engineering and in many other applications³.

One widely investigated Nucleic Acid (NA) editing techniques is known as Clustered Regularly Interspaced Short Palindromic Repeats (CRISPR) genome/NA editing. CRISPR utilizes guide RNA (gRNA)-directed Cas nucleases (hereinafter referred to as CRISPR-Cas) to induce double-strand breaks (DSBs) in the treated genome, has shown promising preliminary results as an approach for definitively curing a variety of genetic disorders¹. Additional genome editing techniques that are induced by engineered nucleases, including, but not limited to, are Zinc finger nucleases (ZFNs), transcription-activator like effector nucleases (TALEN), and meganucleases.

Several methodologies have been developed to detect off-target activity in an unbiased manner⁷. The unbiased methods can detect unintended cleavage sites on the whole genome level, without the need for predetermined focus. One such approach is GUIDE-seq (Genome-wide, Unbiased Identification of DSBs Enabled by Sequencing)⁸. GUIDE-seq relies on the introduction of short tag DNA sequence that is transfected into the cells and integrated at the Cas9-induced DSB via NHEJ. The subsequent sequencing of tag-adjacent regions identifies potential off-target sites. Other Unbiased approaches include CIRCLE-seq⁹, SITE-seg¹⁰, and DISCOVER-Seq¹¹, among others. There are various bioinformatics analysis tools^{20, 21, 22, 23, 24, 25, 26, 27, 28, 29}, previously suggested for estimation of genome-editing activity rates, based on sequencing data of the edited genome-sequences.

Other known in the art tools utilize a so called treatment (Tx) vs. mock (M) approach (also referred to herein interchangeably as Tx vs. M) in order to assess the rates of gene editing events/indels at given sites introduced by CRISPR to the genome treated sequences (Tx). This is achieved by processing/sequencing of treated genome sequences (Tx), which were treated by CRISPR as well as processing/sequencing of comparable/similar none treated genome sequences, mock (M), e.g. which are similar to the treated sequences prior to their treatment by CRISPR), and comparing the rates of indels found at the given sites in both the treated and mock specimens/sequences.
For example, CRISPResso (see e.g. CRISPResso1/2^{31, 32}) and AmpliCan³³, are two widely used tools which utilize Tx vs M data to assess INDEL activity rates of the genome editing treatment by subtract the separately inferred INDEL rates from each of the treatment (Tx) and mock (M) specimens.

Conventional methods for de novo translocation detection, AMP-Seq^{17, 8} (Anchored Multiplex-PCR sequencing), High-Throughput, Genome-wide, Translocation Sequencing (HTGTS)¹⁸, and Uni-Directional Targeted Sequencing (UDiTaS)¹⁹, identify translocations which involve particularly selected DSB sites (on-target or off-target) which might be inflicted by genome editing using site-directed nucleases.

### GENERAL DESCRIPTION

There is a need in the art for a technique capable of determination of editing activity, particularly off-target/adverse editing activity of Nucleic Acid (NA)/genome techniques. Further, there is a need in the art for a novel technique capable of quantifying the occurrence off-target activities/adverse effects over a broad spectrum of possible editing events (e.g. double strand breaks (DSBs) which might be introduced to various NAs subjected to editing by particular one or more NA editing procedures, indels of various types or classes). Also, there is a need in the art for a novel technique enabling identification and possibly quantification of translocations occurring due to NA editing processes/procedure.

Present NA editing techniques, such as CRISPR-based genome editing systems, generally lack the required genome editing specificity and accuracy, which is required for in various NA/genome editing applications. The lack of specificity is manifested for example by off-target activity of genome editing systems, which may in turn introduce unwanted and un-stable/predictable genome editing events at undesired sites/loci. This presents a major drawback for the usability of gene editing systems for various applications since the unwanted genome/NA editing events may result in unwanted mutations and unwanted genomic structural variations, such as translocations.

In this regard, it might be noted that NA editing systems, such as CRISPR-Cas endonucleases, did not naturally evolve to function as a highly specific gene-editing mechanism, certainly not in the context of mammalian genomes. Using these bacterial nucleases in mammalian, plant, and other types of cells often entails off-target activity, leading to unintended DNA breaks at other sites in the genome with only partial complementarity to the gRNA sequence².

To bring CRISPR technology, or any other Nucleic Acid (NA) editing techniques (e.g. with engineered nuclease), to safe broader use in the clinic or for genome editing in mammalian, crop engineering, synthetic NA editing, and/or other applications^{3, 4, 5, 6}, it must be highly active at the on-target site (which is the genome site/locus intended for nucleases by the CRISPR mechanism) and have minimal off-target editing adverse effects (i.e. having minimal editing effects at off-target genome sites/loci being sites/loci other than the on-target sites/loci).

Potential off-target activities NA editing procedures, such as CRISPR-based procedures, present major pitfalls for using this technique of genome editing, due to potentially unwanted NA editing events which may lead for example to genome instability such as resulting mutations (e.g. due to unwanted indels) and/or NA/genomic structural variations (e.g. due to unwanted translocations).

For example, the Cas9 endonuclease can create DSBs at undesired off-target locations, even in the presence of mismatches. This may lead to adverse effects such as indels and translocations.

It should be noted that in the description herein below the following terms/phrases should be understood to encompass at least as follows:
The term Nucleic Acid (NA) is used herein to designate any type of Nucleic Acid to which NA editing procedure may be applied according to existing or future NA editing techniques/procedure. This may encompass DNA, RNA from biological or synthetic origins/source, and may also include Nucleic Acids composed of 4 bases as in biological NAs or NAs composed of different number of base types (e.g. 6 as possible with NAs of synthetic origin).
Accordingly, the term source or NA source, is used herein to designate any type of Nucleic Acid origin, and may encompasses biological source (e.g. living organism, or tissue or sample thereof acquired in-vivo or in-vitro), and/or a synthetic source of NA artificially produced by NA synthesizer.
The phrases NA editing techniques/procedure is used herein to designate any type of existing or future NA editing procedures. Widely known such NA procedures include the CRISPR based NA editing techniques which may utilize various types of CRISPR Associated proteins (CAS), such as CAS9 or other such Engineered nucleases proteins. In this regard, it would be appreciated that the technique of the present invention is aimed at determining/assessing effects or adverse effects of NA editing procedure, and is not limited to the specific type, existing or future, NA editing procedure to be examined thereby.
The terms site(s) and locus/loci are used herein interchangeable to designate respective NA sequence(s) identifiable by their suffix- and prefix-sub-sequences.
In turn the terms suffix- and prefix-sequences (or -sub-sequences) of an NA sequence/site are used herein to designate respective parts of the NA sequence/site, having predetermined respective sequence of a certain number of bases appearing near the beginning and end of the respective site.
The terms forward- and reverse-primer sequences are used herein with interchangeably with the respective terms suffix- and prefix-sequences and should be understood to acquire the respective similar meanings. In this regards it should be noted that although often it is convenient to use of the forward- and reverse-primer sequences the similar sequences as those of the respective forward- and reverse-primer molecules which are used in the amplification of the respective site, this is not essential and other forward- and reverse-primer sequences near the respective ends of the site may be used as forward- and reverse-primer sequences for the purpose of sequencing data analysis of according to the present Invention.
The phrase INDELs is used herein to designate insertion or deletion of bases in an NA sequence.
The phrase TRANSLOCATIONs is used herein to designate structural variations which may be caused by NA editing and which are characterized by fusion of fragments of NA sequences of different sites. Although the phrase typically refers to a chromosomal abnormality, whereby there's a break in the chromosome, one particular chromosome, and that chromosome will then fuse to a different chromosome. However, it should be noted that in the scope of the present invention as described herein, the phrase translocation, not necessarily refers to intra-chromosomal abnormality, but is also intended and used to encompass large deletions within the same chromosome, by which two sites of the same chromosome may be fused together.

Considering the potential off-target activities NA editing procedures, it might be noted that it is possible to design different NA editing procedures (e.g., different CRISPR-based procedures) to targeting the same or similar on-target location, and having the same/equivalent required NA editing effect on the on-target location, but having different adverse effects or different off-targets' activities and/or different probabilities to induce these different off-targets' activities. This might be achieved for instance by different designs of the guide RNA of NA editing procedures, such as CRISPR-based procedures) to target/bind the similar on-target location, but with different likelihoods to bind to undesired off-target locations.

To this end, one approach to overcome these drawbacks (e.g. the lack of specificity) of general NA editing procedures, rely on the ability to accurately inspect the on/off target effects of one or more NA/gene editing procedures (e.g. having different guide RNAs) designed for a particular NA editing application (e.g. to a particular on-target activity) to determine their respective specificities to the required on-target location, or otherwise their respective off-target activities (adverse effects). This will enable selection of a proper NA/gene editing procedure, for a given NA editing application, which has sufficiently high specificity and/or, sufficiently negligible adverse effects, in terms of their probabilities of occurrence by the NA editing or in terms of the significance of the adverse effects introduce thereby (e.g. according to the expected effects of editing in the particular off-target loci affected thereby).

To achieve that there is a need for a technique to accurately and reliably assess the performance of NA/genome editing procedures, and particularly to be capable of accurately identifying and quantifying the resulting off-target activities of such NA/genome editing procedures, when applied to NA sequences of the required NA editing application.

However, the conventional techniques for assessing the performance of gene editing systems are lacking in various respects.

Conventional ‘unbiased’ techniques, such as GUIDE-seq⁸ CIRCLE-seq⁹, SITE-seq¹⁰, and DISCOVER-Seq¹¹, have been established identifying potential genome-wide off-target activity caused by NA editing procedures such as CRISPR-Cas9 genome editing. These techniques are used to detect/determine potential off-target activity, and provide data indicative of potential off-target sites of a genome editing procedure. These techniques are however note capable of validating or quantifying the actual occurrence of adverse effects in those potential off-target sites by the gene editing procedure and require further validation and/or quantification of the actual occurrence of such potential effects by techniques (for example by utilizing targeted amplicon sequencing with primers designed for the reported genomic loci at which adverse effect is expected (e.g., rhAmpSeq^{12, 13}). These techniques are not capable of identifying translocations and not quantitatively measuring any other editing activity of genome editing procedures, but can identify potential off-target locations of genome editing in an unbiased manner.

The off-target activity identification obtained from bioinformatics analysis tools^{20, 21, 22, 23, 24, 25, 26, 27, 28, 29}, which rely solely on the sequencing treated/edited genome/DNA-sequences (e.g. NGS data of the edited genome), is also deficient and inaccurate. This is because the off-target activities identified by such tools may be obscured by sequencing errors introduced to the NGS data. More specifically, in the process of generating sequencing data for the edited genome/DNA-sequences, a number of errors are/maybe generally introduced by the sequencing it-self, and may then be represented in the raw sequencing data. These errors/events may come from various sources (e.g. the sequencing platform, polymerase errors, and library preparation buffers such as, base oxidation)³⁰). These errors from the sequencing itself can often occur near or at the site of the CRISPR-induced DSB, thus limiting the accuracy of bioinformatics analysis tools aimed to quantify off-target events with low activity rates. This is because it is generally difficult to distinguish sequencing/NGS errors from actual edit events.

The conventional tools based on the so called treatment (Tx) vs. mock (M) approach, such as CRISPResso and ampliCan, are limited by their ability to only roughly assess adverse effects of only INDEL category. These methods are based on a simple subtraction of the inferred INDEL activity rates of the mock (M) from those in the treatment (Tx). These techniques are incapable of providing any statistical assessment/determination of whether an adverse effects of each certain type, which is observed in the treatment (Tx), actually occurred due to an edit event, and are also not adapted for segregating the INDEL activity rate inferred thereby into different indel types. Moreover these techniques also do not provide any statistical evaluation or confidence intervals for the inferred rates of INDEL activity.

CRISPResso is for example deficient in that it provides only a combined Tx vs M indel activity rates at a given site of the treated and mock sequences, while not being stratified into different indel types which may occur at said given site. This may yield a problematic and inaccurate assessment of the indel activity rates of the CRISPR, particularly for example in cases where a greater incidence of indel events of a certain type (e.g. which may be unique caused by the gene-editing/CRISPR treatment), are masked by other types of indel events/pseudo-events at the given site, which may appear in relative abundances at the given site, due for example to sequencing artifacts or other/natural processes.

Although, ampliCan does attempt to separately present the different modification/indel types produced by the genome editing, it performs a simple background subtraction for each modification type (Tx-M). However also this technique is incapable of providing any statistical assessment/determination of whether an adverse effects of each certain type, which is observed in the treatment (Tx), actually occurred due to an edit event. This technique, is also incapable of providing a statistical estimate of the inferred rates or of the obtained differences for each modification, and the simple background subtraction may often lead to abused results (such as negative inferred rates of certain indel types in the treatment. Furthermore, ampliCan accounts for different modifications as belong to the same indel type only if they are identical (same position in the reference sequence and with identical base pairs). Namely, ampliCan does not aggregate modifications with similar characteristics, such as insertions at the same reference position and with identical lengths, but with different base pairs. Thus, ampliCan is sensitive to ‘noisy’ indels which originated by amplification, PCR or sequencing errors.

Moreover, additional deficiency of ampliCan's technique is that it is only known/demonstrated to perform with singleplex PCR amplification. This means that the technique is limited to the detection of indels at only one site (typically the on target site) There are, in particular, no performance indications as related to detecting off-target activity by AmpliCan.

Translocations represent a group of possible adverse consequences of off-target editing, that can be particularly devastating even when occurring at low frequencies. For instance deleterious genomic structural variation events are structural variation that can lead to the onset of several human disease conditions including many types of cancer, infertility, and other acquired genomic disorders^{14, 15}. Translocations, such as Chromosomal translocations and large deletions, are structural variation that can arise when on-target/off-target or off-target/off-target cut-sites (on the same or on different Chromosomes) fuse as a result of DSBs at both loci¹⁶.

Therefore, translocations and other adverse/undesired structural variation effects of NA editing require thorough investigation to understand their prevalence, characteristics, and the conditions of an NA editing procedure promoting or repressing their formation, prior to the administration of the NA editing procedure for any particular use.

Conventional techniques, such as those directed for de novo translocation detection (e.g. the above mentioned AMP-Seq^{17, 8}, HTGTS¹⁸, and UDiTaS) address a fixed predetermined list of potential events on one side. For example, in UDiTas genomic sequences are amplified by one sequence-specific primer, targeted to a specific genomic locus, on one side, and a second tagmentation-mediated primer on the other side. Thus, this technique allows for identifying any edit events, including structural variations, as well as any translocation partners of a specific off-target site, at a particular predetermined genomic location.

DSBs are important components of the process leading to the translocation, and therefore there is a need in the art for a technique that is capable of investigate all the possible pairwise translocation events that can take place as a result of an NA editing procedure associated with set of potential off-target sites (e.g, potential off-target sites which may be identified for example by techniques such as GUIDE-seq, Circle-Seq, etc).

However, the conventional de novo translocation detection techniques are inherently deficient in this respect, due to their limited ability to identify only those translocations/genome-editing events, which involve DSB at one or more of the limited set of selected DSB sites which are monitored thereby. This is generally insufficient since off-target gene editing events may occur also at locations other that the particular limited set of DSB sites monitored by such techniques, and particularly translocations can involve various combinations of off-target loci/sites or may occur between the off-target loci and spontaneous breaks. It is also important to note that most or all of these existing techniques are also deficient in that they are designed or not suitable for processing sequencing results from multiplexed amplification, by which multiple potential on and off-target effects may be simultaneously observed.

The present invention provides a novel technique for identification/detection and/or quantification of adverse effects actually occurring due to an NA editing procedures, such as translocations and indel types. Accurate determining of the adverse effects of NA/genum editing is essential in the field of genetics and genomics to support the assessment of the effectiveness and side effects of a genome editing protocol/procedure. The techniques of the present invention solve the deficiencies of the conventional techniques and facilitates accurate statistical determination/assessment of the actual occurrence of various respective types of those adverse effects over the entire spectrum of NA sequences/sites that are expected to be potentially affected by each NA editing procedure. The invention thus provides the ability to assess the specificities and/or adverse-effects of various NA/genome editing procedure (e.g. assess their accuracy, precision, specificity and/or adverse effects in the context of a given genome editing application) and to thereby select the most suitable NA editing configuration/procedure for a given NA/genome editing application, having the high specificity and reduced or insignificant adverse effect for that application.

The technique of the present invention is based on the so called treatment (Tx) vs. mock (M) approach and utilizes advanced modeling for the analysis of Tx vs M multiplex-amplification/PCR data to obtain accurate measurement (determination and/or quantification) of off-target activity of an NA editing procedure with the ability to accurately determine indel types as well as translocations.

The technique of the present invention facilitates accurate statistical detection/determination of indel types occurring due to an NA edit procedure, as well as, statistical quantification thereof by utilizing a statistical model based on a comparative statistical model approach for the indel quantification. Alternatively, or additionally, the technique of the present invention facilitates accurate detection, quantification and statistical assessment of observed translocation. This is achieved by the detection of alternative cut-sites in off-target loci and by proper modeling of the identified alternative cut sites in the mock and treatment data. The technique of the present invention is suitable of analysis of multiplex-PCR and NGS data, based on which it can detect various types of indels and structural variations including deletions and insertions (indels) as well as translocation events occurring in an NA editing procedure, and to output data indicative of the types of adverse effect actually occurring due to the an NA edit procedure (as statistically determined) and possibly also the off-target activity rates of those actually occurring adverse effects as well as confidence intervals for the inferred off-target activity rates.

The technique of the present invention is advantageous inter-alia in that it enables for a comprehensive evaluation of all possible indels and/or translocations among the predicted off-target sites addressed by a single multiplex-PCR, while obviating a need to perform a multiple additional experiments or PCR amplifications. In other words, the technique of the present invention facilitates to determine various translocation events involving the potential off-target sites of an NA editing procedure with as little as a single multiplex amplification of the edited NA sequences.

Thus according to one broad aspect of the present invention there is provided a method for determining effects of Nucleic Acid (NA) editing procedure. The method includes:
Receiving sequencing data resulting from sequencing of multiplexed amplifications products/amplicons of a first and second collections of NA sequences, such that the sequencing data is indicative of pluralities of reads, R_{Tx} {rᵢ^{Tx}} and R_{Mc}={rⱼ^{Mc}}, of the multiplexed amplifications' products/amplicons from each of the first and second collections (typically e.g. in the order of for example 10,000 to 100,000 read or more per each collection), whereby:
   The first and second collections of NA sequences originate from the same NA source, such that the first collections of NA sequences is an edited collection of NA sequences from said NA source to which a certain NA editing procedure is applied, and the second collection is a control (mock) collection of NA sequences of said NA source to which said NA editing procedure is not applied;
   The multiplexed amplifications of the edited and control collections are conducted with similar set of a plurality of primer molecule types {PRₜ};
   The set of a plurality of primer molecule types {PRₜ} is designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites;
Processing said sequencing data by a processor for constructing, per each particular type of adverse effect of one or more types of possible adverse effects of said NA editing procedure, a statistical model of occurrence of said type of adverse effect by the NA editing procedure, and applying said statistical model to said sequencing data to statistically determine actual occurrence of said type of adverse effect by the NA editing procedure; and
Outputting data indicative of whether said each type of adverse effect by actually occurs due to the NA editing procedure, to thereby enable determination of adverse effects of the NA editing procedure.

In some implementations the said one or more types of adverse effect are classified to one or more classes of adverse effects, each class being characterized by the one or two involving sites [λₘ₁,λₘ₂], with which adverse effects of the class are involved, whereby each class belongs to one of two categories of adverse effects:
Category 1 (INDELs): adverse effects involving one site [λₘ₁=m₂] where m1=m2; and
Category 2 (TRANSLOCATIONs): adverse effects involving two sites [λₘ₁,λₘ₂] where m1≠m2.
   The processing of the sequencing data for the particular type of adverse effect may include carrying out the following operations a. to d. for at least one of the two categories of adverse effects:
a. Providing a template statistical model corresponding to the category of said particular type of adverse effect;
b. Processing the sequencing data according to the class [λₘ₁,λₘ₂] of said particular type of adverse effect to determine or assess respective ‘collective’ counts N_{Tx} and N_{Mc} of reads of amplicons which are associated with the involving sites [λₘ₁,λₘ₂] of said class in the sequencing data of each of the edited and control collections;
c. Processing the sequencing data according to the particular type of adverse effect to determine or assess respective ‘affected’ counts n_{Tx} and n_{Mc} of reads of amplicons in which said particular type of adverse effect is observed;
d. Applying said template statistical model to the respective ‘collective’ counts N_{Tx} and N_{Mc} of reads of amplicons which are associated with the involving sites [λₘ₁,λₘ₂] in the edited and control collections and to the respective ‘affected’ counts n_{Tx} and n_{Mc} of reads of amplicons in which said particular type of adverse effect is observed in the reads of amplicons which are associated with the edited and control collections; and
   The method thereby statistically determines whether said particular type of adverse effect is affected by the NA editing procedure.

According to some embodiments of the present invention the method also includes one or more of the following preliminary operations:
providing a first and second collections of NA sequences originated from the same NA source, whereby the first collection is an edited collection of NA sequences from said NA source to which a certain NA editing procedure was applied, and the second collection is a control (mock) collection of NA sequences of said NA source to which said NA editing procedure was not applied;
providing target data indicative of expected editing sites {λₘ}₁^{M} of the NA editing procedure including at least one on-target site {λ₁} and one or more off-target sites {λₘ}₂^{M}, where λₘ represents an off-target or on-target site indexed m and M is a number of the expected on-target and off-target sites;
applying multiplexed amplifications to the edited and control collections respectively, and thereby obtaining respective amplified products/amplicons of said edited and control collections, whereby the multiplexed amplifications of the edited and control collections are conducted with similar primer molecule types;
sequencing the multiplexed amplifications products/amplicons of said edited and control collections to obtain sequencing data indicative of pluralities of reads, R_{Tx}={rᵢ^{Tx}} and R_{Mc}={rⱼ^{Mc}}, of said multiplexed amplifications' products/amplicons from each of the edited and control collections.

The sequencing data may then be processed by a processor for constructing, per each particular type of adverse effect of one or more types of possible adverse effects of said NA editing procedure, a statistical model of occurrence of said type of adverse effect by the NA editing procedure, and applying said statistical model to said sequencing data to statistically determine actual occurrence of said type of adverse effect by the NA editing procedure. The data indicative of the of whether said each type of adverse effect by actually occurs due to the NA editing procedure, may be output enable determination of safety of the NA editing procedure.

According to some embodiments the processing further comprising utilizing the statistical model to quantify the types of adverse effects actually affected by the NA editing procedure by determining rates of occurrence thereof by the NA editing procedure, and a statistical confidence intervals for said rates.

According to some embodiments the one or more types of adverse effect are classified to one or more classes of adverse effects, each class being characterized by the one or two participating sites [λₘ₁,λₘ₂], with which adverse effects of the class are associated, whereby each class belongs to one of two categories of adverse effects:
Category 1 (INDELs): adverse effects involving one site [λₘ₁₌ₘ₂] where m1=m2; and
Category 2 (TRANSLOCATIONs): adverse effects involving two sites [λₘ₁,λₘ₂] where m1≠m2.

According to some embodiments the processing said sequencing data for the particular type of adverse effect includes obtaining the statistical determination of the occurrence of said particular type of adverse effect by the NA editing procedure in the tested cell types or in related samples or clinical material.

According to some embodiments the multiplexed amplifications of the edited and control collections are conducted utilizing respective multiplex PCR processes with a similar selected set of primer molecule types{PRₜ}. The method includes providing the selected set of a plurality of primer molecule types {PRₜ} including primer molecule types selected according to said target data, such that the plurality of primer types {PRₜ} comprise, or constitutes of, matched pairs (PRM⁺ₘ, PRM⁻ₘ) of forward PRM⁺ₘ and reverse PRM⁻ₘ primer molecule types {(PRM⁺ₘ, PRM⁻ₘ)}₁^{M} ∈{PRₜ} suitable for amplification of said on-target and off-target sites {λₘ₁}₁^{M} in the edited and control NA collections. These may for example be used in a One-Step-Amplification or in the 1^{st} step of a Two-Step-Amplification.

In some implementations the processing includes a preliminary preprocessing of the sequencing data for adjusting said reads of said multiplexed amplifications' products/amplicons from each of the edited and control collections by carrying out at least one of the following:
trimming of sequencing adapters from said reads,
merging pair-end reads, and
filtering out low-quality reads.

According to some embodiments the method is adapted for determining at least one indel type T of said one or more of types of adverse effect of the NA editing procedure, which belong to the Category 1 of adverse effects that is associated with INDEL activity of said NA editing procedure, and which belong to at least one class of adverse effects associated with a respective site/locus of interest λₘ₁.

According to some embodiments the processing of the sequencing data includes matching reads of said sequencing data to the site/locus of interest λₘ₁ by carrying out the following:
a. providing reference data indicative of at least one reference NA sequence of the at least one respective site/locus of interest λₘ₁ for which INDEL activity of said NA editing procedure is to be assessed;
b. utilizing a ‘collective’ count match condition of said template statistical model, to identify matched reads of the multiplexed amplifications products/amplicons that match the reference NA sequence of the site/locus of interest λₘ₁ in the reference data; and thereby obtaining for the site of interest, λₘ₁, respective collections L_{Tx}(λₘ₁) and L_{M}(λₘ₁) of matched reads, in the sequencing data of the edited and control collections respectively.

The ‘collective’ count match condition of the template statistical model of Category 1 of adverse effects may for example be satisfied for a read in case the prefix and suffix and regions of the read match prefix PRS⁺ₘ and suffix PRS-m primer sequences (PRS⁺ₘ₁, PRS⁻ₘ₁) of the respective site of interest λₘ₁.

In some implementations the sizes of the respective collections L_{Tx}(λₘ₁) and L_{M}(λₘ₁) are used as the ‘collective’ counts N_{Tx} and N_{Mc} of reads of amplicons which are associated with said at least one class of adverse effects involving the site λₘ₁ observed in the sequencing data of the corresponding edited and control collections.

In some embodiments the processing of the sequencing data by includes segregating the collections L_{Tx}(λₘ₁) and L_{M}(λₘ₁) of reads matching the site of interest λₘ₁, to form at two sub-collections L_{Tx}(λₘ₁,T) and L_{M}(λₘ₁,T) of reads presenting a certain type T of indel observed in the matched reads from the sequencing data of the edited and control collections respectively. Each indel type T is characterized by at least one of:
a size/length τ of bases introduced-to or deleted-from the matched read relative to the reference NA sequence of the site/locus of interest λₘ₁, and
a position i along the site of interest λₘ₁ at which said bases are introduced-to or deleted-from; and

The segregating may include carrying out the following:
(a) aligning the matched reads in the collections L_{Tx}(λₘ₁) and L_{M}(λₘ₁) to the reference NA sequence of the site of interest λₘ₁, and
(b) identifying gaps in the aligned matched reads whereby each gap representing an indel and at least one of a position i and a length τ of the gap represents a type T of said indel; and
(c) respectively aggregating the aligned matched reads of the collections L_{Tx}(λₘ₁) and L_{M}(λₘ₁), to form the corresponding sub-collections L_{Tx}(λₘ₁,T) and L_{M}(λₘ₁,T) of reads, respectively presenting observations of said certain type T of indel, in the aligned matched reads of the sequencing data of the corresponding edited and control collections, whereby said aggregating comprises matching the identified gaps in the aligned matched reads with properties of a gap representing said certain type T of indel based on an ‘affected’ count match condition of the template statistical model of Category 1 of adverse effects, whereby said ‘affected’ count match condition of the template statistical model of Category 1 is satisfied upon fulfillment of a predetermined set one or more of the following conditions:
   i) the position i of an identified gap in an aligned matched read is similar to a position î of the gap in said type T of indel;
   ii) a size τ of the gap of an identified gap in an aligned matched read is similar to a size \hat{ }τ of the gap in said type T of indel;
   iii) a nucleotide base sequence in the gap of an identified gap in an aligned matched read has a degree of similarity with a nucleotide base sequence of said type T of indel above a certain threshold.

In some embodiments the indel type T is characterized by both said size/length τ of bases and said position i.

In some embodiments the sizes of said respective sub-collections L_{Tx}(λₘ₁,T) and L_{M}(λₘ₁,T) are used as the ‘affected’ counts n_{Tx} and n_{Mc} of reads of amplicons, in which said particular type T of adverse effect is observed.

In some embodiments the template statistical model provided for the INDEL activity of said NA editing procedure comprises a statistical classifier comprising a Maximum A Posteriori (MAP) estimator. In this case the method may include provision of probability reference data indicative of prior probabilities P(edit) and P(no edit) of occurrence of edit and no-edit associated with observance of said indel type T. The prior probabilities P(edit) and P(no edit) may be complementary P(edit)=1−P(no edit).

In some embodiments the prior probabilities P(edit) and P(no edit) are provided as functions depending on a distance between a position i along the site of interest λₘ₁ at which said indel of the type T is observed and to an expected cut-site position i₀ of the NA editing procedure at the site of interest λₘ₁. For example in some embodiments the prior probabilities the prior probabilities within a predetermined window of distances between the position i and the expected cut-site position i₀ are set with fixed predetermined probabilities (e.g. trivial priors of 0.5), and outside said range decrease in order, according to distance |i−i₀| between the position i and the expected cut-site position.

Alternatively, the prior probabilities P(edit) and P(no edit) of said MAP estimator may set as fixed trivial probabilities independent of position P(edit)=P(no edit)=0.5, and said MAP estimator thereby functions as maximum likelihood estimator (MLE).

In some implementations, the MAP estimator of said template statistical model is a Bayesian classifier and wherein said applying of said template statistical model to the respective ‘collective’ counts N_{Tx} and N_{Mc} of reads and the respective ‘affected’ counts n_{Tx} and n_{Mc} comprises computing said Bayesian classifier to determining that said NA editing procedure affected an edit causing the indels of type T in case the following is satisfied according to Bayes formula as follows:

P(edit|n_{Tx},n_{M})>P(no edit|n_{Tx},n_{M})⇔P(edit)·P(n_{Tx},n_{M}|edit)>P(no edit)·P(n_{Tx},n_{M}|no edit)

wherein P(edit|n_{Tx},n_{M}) and P(no edit|n_{Tx},n_{M}) are the respective probabilities that an edit hypothesis and a no-edit hypothesis are valid given the observed ‘affected’ counts n_{Tx} and n_{Mc} of indels of type T in the in the edited and control sequences; P(edit) is a prior probability that an observed indel type T was caused by an edit event, and P(no edit) is a complementary prior probability P(edit)=1−P(no edit); P(n_{Tx},n_{M}|no edit) is a probability of observation of the ‘affected’ counts n_{Tx} and n_{Mc} in the edited and control sequences under an assumption that there was no edit causing the ‘affected’ count n_{Tx} observed in the edited sequences; P(n_{Tx},n_{M}|edit) is a probability of observation of the under an assumption that there was an edit causing the ‘affected’ count n_{Tx} observed in the edited sequences.

In some embodiments the template statistical model includes utilizes a hyper-geometric distribution for computing the probability P(n_{Tx},n_{M}|no edit) of observation of the ‘affected’ counts n_{Tx} and n_{Mc} under the no edit assumption.

In some embodiments the template statistical model utilizes a binomial distribution for computing assessing the probability P(n_{Tx},n_{M}|edit) of observation of the ‘affected’ counts n_{Tx} and n_{Mc} under the edit assumption.

To this end in some implementations the method includes provision of reference probability parameter q of said binomial distribution. The reference probability parameter q is indicative of a probability that an observed indel of type T in the reads of the edit and control collections has occurred through an edit event.

The method may be alternatively or additionally be adapted for determining at least one particular type T of translocation (Category 2 of adverse effects) out of said one or more of types of adverse effect of the NA editing procedure. The at least one particular type T of translocation may be characterized as translocation involving both sites of a respective pair of sites/loci of interest λₘ₁, λₘ₂ where m1≠m2.

In such embodiments the processing of the sequencing data may include matching reads of said sequencing data to the pair of sites/loci of interest, λₘ₁ and λₘ₂, by carrying out the following:
(a) providing reference data indicative of at least a pair of reference NA sequences corresponding to said pair of sites/loci of interest, λₘ₁ and λₘ₂, for which TRANSLOCATION activity of said NA editing procedure is to be assessed;
(b) Carrying out the above indicated operation b. by utilizing a ‘collective’ count matching condition of the template model of the Category 2 of adverse effects and to process the sequencing data according to the class [λₘ₁,λₘ₂] of said translocation adverse effect for identifying therein single site partially matching collections of read C_{TX}(λₘ₁), C_{TX}(λₘ₂), C_{Mc}(λₘ₁), C_{Mc}(λₘ₂) of reads of the multiplexed amplifications products/amplicons of the edit and mock collections, each satisfying said ‘collective’ count matching condition, wherein said ‘collective’ count matching condition is indicative of whether a read is at least partially matching to at least one reference NA sequence λₘ₁ or λₘ₂, of the pair of reference NA sequences associated with the pair of sites/loci of interest, λₘ₁ and λₘ₂, in the reference data; whereby said sizes of said single site partially matching collections {C_{TX}(λₘ₁), C_{TX}(λₘ₂)}, {C_{Mc}(λₘ₁),C_{Mc}(λₘ₂)} are indicative of the respective ‘collective’ counts N_{Tx} and N_{Mc} of reads of amplicons which are associated with at least one of the involving sites [λₘ₁,λₘ₂] in the sequencing data of each of the edited and control collections; and
(c) Carrying out the above indicated operation C. by processing of the sequencing data according to the characteristics of the particular type T of translocation to determine or assess said respective ‘affected’ counts n_{Tx} and n_{Mc} of reads of amplicons, in which a translocation involving fusion of both site [λₘ₁,λₘ₂], are observed, by utilizing an ‘affected’ count matching condition of the template model of the Category 2 and to identifying dual site partially matching collections C_{TX}(λₘ₁,λₘ₂), C_{Mc}(λₘ₁,λₘ₂) of reads of the multiplexed amplifications products/amplicons of the edit and mock collections, each satisfying said ‘affected’ count matching condition, wherein said ‘affected’ count matching condition is indicative of whether a is at least partially matching to both the reference NA sequence λₘ₁ or λₘ₂, of the pair of reference NA sequences associated with the pair of sites/loci of interest, λₘ₁ and λₘ₂, in the reference data; whereby said sizes of said dual site partially matching collections C_{TX}(λₘ₁,λₘ₂), C_{Mc}(λₘ₁,λₘ₂) are indicative of the respective ‘affected’ counts n_{Tx} and n_{Mc}.

In some implementations the ‘collective’ counts N_{Tx} and N_{Mc} are estimated based on respective sizes of the following pairs of single site partially matching collections [|C_{TX}(λₘ₁)|, |C_{TX}(λₘ₂)|], [|C_{Mc}(λₘ₁)|, |C_{Mc}(λₘ₂)|], and said ‘affected’ counts n_{Tx} and n_{Mc} are determined based on respective sizes of the dual site partially matching collections |C_{TX}(λₘ₁,λₘ₂)| and |C_{Mc}(λₘ₁,λₘ₂)| obtained from the reads of the edit and control collections respectively. For example the ‘collective’ counts N_{Tx} and N_{Mc} may be estimated as respective averages of said respective sizes of the pairs of single site partially matching collections such that: N_{Tx}=|C_{TX}(λₘ₁)|, |C_{TX}(λₘ₂)|> and N_{Mc}=<|C_{Mc}(λₘ₁)|, |C_{Mc}(λₘ₂)|>; and the ‘affected’ counts n_{Tx} and n_{Mc} are determined as the respective sizes of the dual site partially matching collections such that n_{Tx}=|C_{TX}(λₘ₁,λₘ₂)| and n_{Mc}=|C_{Mc}(λₘ₁,λₘ₂)|. In a particular example the ‘collective’ counts N_{Tx} and N_{Mc} are estimated as respective geometrical averages of said respective sizes of the pairs of single site partially matching collections.

In some embodiments the method includes associating each read r, which presents the particular type of translocation between sites λₘ₁ and λₘ₂, with one of the following four possible translocation species S according to the following translocation matching conditions {DS1 to DS4}:
DS1: Pfx(r) [symbol] F(λₘ₁)∧Sfx(r) [symbol] Rev(R(λₘ₂)) or a reverse-complement thereof: Pfx(r) [symbol] R(λₘ₂)∧Sfx(r) [symbol] Rev(F(λₘ₁));
DS2: Pfx(r) [symbol] F(λₘ₂)∧Sfx(r) [symbol] Rev(R(λₘ₁)) or a reverse-complement thereof: Pfx(r) [symbol] R(λₘ₁)∧Sfx(r) [symbol] Rev(F(λₘ₂));
DS3: Pfx(r) [symbol] F(λₘ₂)∧Sfx(r) [symbol] Rev(F(λₘ₁)) or a reverse-complement thereof: Pfx(r) [symbol] F(λₘ₁)∧Sfx(r) [symbol] Rev(F(λₘ₂));
DS4: Pfx(r) [symbol] R(λₘ₂)∧Sfx(r) [symbol] Rev(R(λₘ₁)) or a reverse-complement thereof Pfx(r) [symbol] R(λₘ₁)∧Sfx(r) [symbol] Rev(R(λₘ₂));
   here F(λₘ) and R(λₘ) respectively designate the prefix and suffix primer sequences of the respective, PRS⁺ₘ and PRS⁻ₘ, of site λₘ, and Pfx(r) and Sfx(r) respectively designate prefix and suffix of a read r, L denotes a best match (e.g. according to lowest edit distance between the prefix/suffix of the read and prefix/suffix primer sequences of the plurality of sites {λₘ}₁^{M}, and the reverse complements of said primer sequences), and Rev( ) denotes a reverse-complement function of a nucleic acid sequence.

According to some embodiments the primer molecules, which are used for amplification of a site A, in at least one amplification step, include primer molecules each having respective one of the forward and reverse binding NA sequences and a correspondingly respective one the forward and reverse-adapters, as well as primer molecules each having respective one of the forward and reverse binding NA sequences and correspondingly respective one the reverse and forward adapters. This thereby enables sequencing of reads of all four translocation species: A, B, C and D.

According to some embodiments the processing of the sequencing data includes carrying out said matching reads of said sequencing data to the pair of sites/loci of interest, λₘ₁ and λₘ₂, without segregation to the possible translocation species S∈{DS1 to DS4}, by carrying out the following:
Carrying out the identification of the dual site partially matching collections C_{TX}(λₘ₁,λₘ₂), C_{Mc}(λₘ₁,λₘ₂) in (c) by:
   processing reads rᵢ^{Tx}∈R_{Tx} rⱼ^{Mc}∈R_{Mc} of said multiplexed amplifications' products/amplicons of the respective edited and control collections, to determine for each processed read rᵢ^{Tx} or rⱼ^{Mc} whether it satisfies said ‘affected’ count matching condition, whereby the ‘affected’ count matching condition is satisfied in case at least one of the four translocation matching conditions DS1 to DS4 is satisfied by the processed read; and
   upon determining a match of the processed read rᵢ^{Tx} or rⱼ^{Mc} with any one of the four translocation matching conditions DS1 to DS4, including the matched read rᵢ^{Tx} or rⱼ^{Mc} in the respective collection of dual site partially matching collections C_{TX}(λₘ₁,λₘ₂), C_{Mc}(λₘ₁,λₘ₂) according to an origin of said matching read rᵢ^{Tx} or rⱼ^{Mc} from the multiplexed amplifications' products/amplicons of either the edited collection or the control collection respectively; and
Carrying out the identification of the single site partially matching collections C_{TX}(λₘ₁), C_{TX}(λₘ₂), C_{Mc}(λₘ₁), C_{Mc}(λₘ₂) in (b) by:
   processing each read rᵢ of a plurality of the reads rᵢ^{Tx}∈R_{Tx} rⱼ^{Mc}∈R_{Mc} of said multiplexed amplifications' products/amplicons of the respective edited and control collections, to determine whether said read rᵢ satisfies the ‘collective’ count matching condition with any one of the reference sequences of the sites λₘ₁ and λₘ₂; whereby the ‘collective’ count matching condition includes:
   rᵢ^{Tx/Mc}∈C_{TX/Mc}(λₘ) if one of the following is satisfied by the read:

Pfx(rᵢ) [symbol] F(λₘ) or Pfx(λₘ) [symbol] R(λₘ) or Sfx(rᵢ) [symbol] Rev(F(λₘ)) or Sfx(rᵢ) [symbol] Rev(R(λₘ));
and
upon determining a match of the read rᵢ, including the matched read in the respective collection of the single site partially matching collections C_{TX}(λₘ₁), C_{TX}(λm₂), C_{Mc}(λₘ₁), or C_{Mc}(λₘ₂) based on the site λₘ=λₘ₁ or λₘ=λₘ₂ for which the match exists and an origin of said matching read rᵢ from the multiplexed amplifications' products/amplicons of either the edited collection or the control collection respectively.

In some embodiments the processing of said sequencing data includes carrying out the matching of the reads of said sequencing data to the pair of sites/loci of interest, λₘ₁ and λₘ₂, with segregation to translocation species S={S1 to S4}, by carrying out the following for at least specific translocation species Si∈{S1 to S4}:
Carrying out the identification of the dual site partially matching collections C_{TX}(λₘ₁,λₘ₂), C_{Mc}(λₘ₁,λₘ₂) in (c) for said specific translocation species Si by:
   processing each read rᵢ of a plurality of the reads rᵢ^{Tx}∈R_{Tx} rⱼ^{Mc}∈R_{Mc} of said multiplexed amplifications' products/amplicons of the respective edited and control collections, to determine whether said read rᵢ satisfies said ‘affected’ count matching condition, whereby the ‘affected’ count matching condition is satisfied in case a corresponding one DSi of said four translocation matching conditions DS1 to DS4 is satisfied by the processed read;
   upon determining a match of the processed read rᵢ^{Tx} or rⱼ^{Mc}, including the matched read rᵢ^{Tx} or rⱼ^{Mc} in the respective collection of dual site partially matching collections C_{TX}(λₘ₁,λₘ₂), C_{Mc}(λₘ₁,λₘ₂) according to an origin of said matching read rᵢ^{Tx} or rⱼ^{Mc} from the multiplexed amplifications' products/amplicons of either the edited collection or the control collection, respectively; and
Carrying out the identification of the single site partially matching collections CTX(λₘ₁), C_{TX}(λₘ₂), C_{Mc}(λₘ₁), C_{Mc}(λₘ₂) in (b) by:
   processing each read rᵢ of a plurality of the reads rᵢ^{Tx}∈R_{Tx} rⱼ^{Mc}∈R_{Mc} of said multiplexed amplifications' products/amplicons of the respective edited and control collections, to determine whether said read rᵢ satisfies the ‘collective’ count matching condition with any one of the reference sequences of the sites λₘ₁ and λₘ₂; whereby the ‘collective’ count matching condition is satisfied if a reads has a at least partial single site match with a prefix or suffix sequence of any of the sites λₘ₁ and λₘ₂; and upon determining a match of the read rᵢ, including the matched read in the respective collection of the single site partial match collections C_{TX}(λₘ₁), C_{TX}(λₘ₂) C_{Mc}(λₘ₁), or C_{Mc}(λₘ₂) based on the site λₘ=λₘ₁ or λₘ=λₘ₂ for which the match exists and an origin of said matching read rᵢ from the multiplexed amplifications' products/amplicons of either the edited collection or the control collection respectively.

According to some embodiments the template statistical model provided for assessing the TRANSLOCATION activity of said NA editing procedure includes a statistical classifier comprising a hyper geometric tail distribution for computing said probability of occurrence of the adverse effect, and wherein assessing a probability of TRANSLOCATION activity of at least one type T or species S of said translocation types or species is determined by computing the probability of said hyper geometric tail distribution based on the respective ‘collective’ counts N_{Tx} and N_{Mc} type’ counts n_{Tx} and n_{Mc} of reads of amplicons which are associated with the respective translocation type T or species S.

According to another broad aspect of the present invention there is provided a method for determining translocation adverse effects of Nucleic Acid (NA) editing procedure, the method includes:
Receiving sequencing data resulting from sequencing of multiplexed amplifications products/amplicons of a first and second collections of NA sequences, such that the sequencing data is indicative of pluralities of reads, R_{Tx}={rᵢ^{Tx}} and R_{Mc}={rⱼ^{Mc}}, of the multiplexed amplifications' products/amplicons from each of the first and second collections, whereby:
   Said first and second collections of NA sequences originate from the same NA source, such that the first collections of NA sequences are an edited collection of NA sequences from said NA source to which a certain NA editing procedure is applied, and the second collection is a control (mock) collection of NA sequences of said NA source to which said NA editing procedure is not applied;
   the multiplexed amplifications of the edited and control collections are conducted with similar set of a plurality of primer molecule types {PRₜ};
   said set of a plurality of primer molecule types {PRₜ} is designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites; and said set of the plurality of primer molecule types comprises, or constituted by, match pairs (PRM⁺ₘ, PRM⁻ₘ) of forward PRM⁺ₘ and revers PRM⁻ₘ primer molecule types {(PRM⁺ₘ, PRM⁻ₘ)}₁^{M}∈{PRₜ} suitable for amplification of said on-target and off-target sites {λₘ}₁^{M} in the edited and control NA collections;
Processing the sequencing data to identify at least one type or species of translocation adverse effect involving two different sites [λₘ₁,λₘ₂] of the expected on-target and off-target sites of said NA editing procedure, whereby said processing comprises:
Counting reads rᵢ of pluralities of said reads R_{Tx}={rᵢ^{Tx}} and R_{Mc}={rⱼ^{Mc}} of the edited and control collections which satisfy a single site partial match condition with respect to at least one of the two different sites [λₘ₁,λₘ₂] and thereby assessing respective ‘collective’ counts N_{Tx} and N_{Mc} of reads of the edit and control collection in which at least one of the two different sites [λₘ₁,λₘ₂] is involved;
Counting reads rᵢ of the pluralities of said reads R_{Tx}={rᵢ^{Tx}} and R_{Mc}={rⱼ^{Mc}} of the edited and control collections which satisfy a double site match condition DS associated with said type or species of the translocation adverse effect, to determine respective ‘affected’ counts n_{Tx} and n_{Mc} of reads of the edit and control collection satisfying said double site partial match condition DS; and
Statistically determining whether the ‘affected’ count n_{Tx} in the edit collection is observed due to said at least one type or species of translocation adverse effect occurring in the NA editing procedure, by applying a selected statistical distribution model to said ‘collective’ and ‘affected’ counts N_{Tx}, N_{Mc}, n_{Tx} and n_{Mc}.

The selected statistical distribution model may for example be a hyper geometric tail distribution.

In some implementations the double site partial match condition is elected according to the type or according to the specific species of translocation whose probability of occurrence due to edit is to be assessed.

As indicated above in some implementations the respective ‘collective’ counts N_{Tx} and N_{Mc} are estimated based on an average of two counts of reads of each of the respective edit and control collections, which satisfy said single site match condition for at least one of the two different sites [λₘ₁,λₘ₂].

According to yet another broad aspect of the present invention there is provided a system comprising a non-transitory computer readable medium storing instructions executable by a processor, for determining and outputting data indicative of the effects of Nucleic Acid (NA) editing procedure according to any of the methods of the present invention described above and in ore details herein below.

In some embodiments the system includes or is adapted to operate the following:
an input adapted to receive said sequencing data indicative of the results a sequencing of multiplexed amplifications products/amplicons of said first and second collections of NA sequences;
a memory or a section thereof, for storing the respective pluralities of reads, R_{Tx}={rᵢ^{Tx}} and R_{Mc}={rⱼ^{Mc}}, of the multiplexed amplifications' products/amplicons from each of the first and second collections;
a memory or a section thereof, for storing data indicative of the set of the plurality of primer molecule types {PRₜ} used for said multiplexed amplifications in association with the expected editing sites {λₘ}₁^{M} of the NA editing procedure;
a memory or a section thereof, for storing reference data indicative of at least one reference NA sequence of at least one respective site λₘ of the expected editing sites {λₘ}₁^{M} of the NA editing procedure;
a memory or a section thereof, for storing at least one template statistical model corresponding to at least one category of adverse effects; and
a processor for processing said sequencing data based on said reference data and the template statistical model for applying said template statistical model to said sequencing data to statistically determine the occurrence of at least one type of adverse effect of said at least one category, by the NA editing procedure; and
an output for outputting data indicative of the determined occurrence of said at least one type of adverse effect by the NA editing procedure.

In some implementations the system is configured and operable to determine occurrence of one or more of said types of adverse effect, which are associated with at least one category of the two categories of adverse effects:
Category 1 (INDELs): adverse effects involving one site [λₘ₁₌ₘ₂] where m1=m2; and
Category 2 (TRANSLOCATIONs): adverse effects involving two sites [λₘ₁,λₘ₂] where m1≠m2; and

The processor may be adapted to process the sequencing data for each particular type of adverse effect of said one or more of said types of adverse effects of said at least one category by carrying out the following:
a. Retrieving from the reference data stored in said memory or section thereof, reference NA sequences of the one or two sites [λₘ₁,λₘ₂] participating in said particular type of adverse effect;
b. Obtaining the template statistical model corresponding to the category of said particular type of adverse effect;
c. constructing a statistical model for said particular type of adverse effect based on said template statistical model and the reference data, by carrying out the following:
   Processing said sequencing data of the first and second collection to determine or assess respective ‘collective’ counts N_{Tx} and N_{Mc} of reads of amplicons which are associated with the one or two sites [λₘ₁,λₘ₂] participating in said particular type of adverse effect, by matching said amplicons to the reference NA sequences of said one or two sites [λₘ₁,λₘ₂] according to a ‘collective’ count match condition designated by said template statistical model, and respectively counting the reads of amplicons of said first and second collections, which satisfy said ‘collective’ count match condition, to thereby determine or assess the respective ‘collective’ counts N_{Tx} and N_{Mc};
   Processing said sequencing data of the first and second collection to determine or assess respective ‘affected’ counts n_{Tx} and n_{Mc} of reads of affected amplicons in which said particular type of adverse effect is observed, by matching said amplicons to the reference NA sequences of said one or two sites [λₘ₁,λₘ₂] according to an ‘affected’ count match condition designated by said template statistical model, and respectively counting the reads of amplicons of said first and second collections, which satisfy said ‘affected’ count match condition, to thereby determine or assess the respective ‘affected’ counts n_{Tx} and n_{Mc};
d. Determining whether said particular type of adverse effect occurs due to the NA editing procedure, by applying said statistical model to the sequencing data, whereby said applying comprises:
   utilizing a statistical classifier of the template statistical model that corresponds to the category of said particular type of adverse effect; and
   computing statistical classifier based on said ‘collective’ counts N_{Tx} and N_{Mc} and said ‘affected’ counts n_{Tx} and n_{Mc} to determine whether said particular type of adverse effect occurs due to the NA editing procedure.

According to yet another broad aspect there is provided a system for determining and outputting data indicative of the effects of Nucleic Acid (NA) editing procedure. The system may include:
an input adapted to receive sequencing data indicative of respective read results comprising of pluralities of reads, R_{Tx}={rᵢ^{Tx}} and R_{Mc}={rⱼ^{Mc}} obtained by sequencing of multiplexed amplifications products/amplicons of first and second collections of NA sequences, whereby the first collection is an edited collection of NA sequences to which a certain NA editing procedure was applied, and the second collection is a control collection of NA sequences to which said NA editing procedure was not applied;
a memory or a section thereof, for storing the respective pluralities of reads, R_{Tx}={rᵢ^{Tx}} and R_{Mc}={rⱼ^{Mc}}, of the multiplexed amplifications' products/amplicons from each of the first and second collections;
a memory or a section thereof, for storing data indicative of the set of the plurality of primer molecule types {PRₜ} used for said multiplexed amplifications of expected editing sites {λₘ}₁^{M} of the NA editing procedure;
a memory or a section thereof, for storing reference data indicative of at least one reference NA sequence of at least one respective site λₘ of the expected editing sites {λₘ}₁^{M} of the NA editing procedure;
a memory or a section thereof, for storing at least one template statistical model corresponding to at least one category of adverse effects; and
a processor for processing said sequencing data based on said reference data and the template statistical model for applying said template statistical model to said sequencing data to statistically determine occurrence of at least one type of adverse effect of said at least one category, due to the NA editing procedure; and
an output for outputting data indicative of the statistically determined occurrence of at least one type of adverse effect by the NA editing procedure.

The processor of the system may be is adapted to process said sequencing data for said type of adverse effect by carrying out the above indicated operations a. to d.

In some embodiments the systems described above may be configured and operable for determining said probability of occurrence for adverse effects of one or both of the following categories:
Category 1 (INDELs): adverse effects involving one site [λₘ₁₌ₘ₂] where m1=m2; and
Category 2 (TRANSLOCATIONs): adverse effects involving two sites [λₘ₁,λₘ₂] where m1≠m2.

In some implementations the systems described above may include a sequencing utility capable of sequencing of the multiplexed amplifications products/amplicons of the first and second collections of NA sequences. In such implementations the input may be connected/connectable to the sequencing utility for receiving said sequencing data therefrom.

In some implementations of the methods and systems of the invention the sequencing may be conducted utilizing NGS sequencing techniques.

According to further another broad aspect of the present invention there is provided a kit for determining effects of a NA editing procedure. The kit includes:
a set of a plurality of primer molecule types {PRₜ} designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites; and
a system according to the present invention as described above, and in more details below.
For instance, the system of the kit may include a non-transitory computer readable medium storing instructions executable by a processor, for determining and outputting data indicative of the effects of Nucleic Acid (NA) editing procedure according to any of the methods of the present invention described above and in ore details herein below.

In some embodiments of the kit, the set of the plurality of primer molecule types {PRₜ} include matched pairs (PRM⁺ₘ, PRM⁻ₘ) of forward PRM⁺ₘ and reverse PRM⁻ₘ primer molecule types {(PRM⁺ₘ, PRM⁻ₘ)}₁^{M}∈{PRₜ} suitable for amplification of said on-target and off-target sites {λₘ}₁^{M}.

In some embodiments the primer molecules, which are used for amplification of a site λₘ in at least one amplification step, include primer molecules each having respective one of the forward and reverse binding NA sequences and a correspondingly respective one the forward and reverse-adapters, as well as primer molecules each having respective one of the forward and reverse binding NA sequences and correspondingly respective one the reverse and forward adapters thereby enabling sequencing reads of all four translocation species: A, B, C and D.

According to further yet another broad aspect of the present invention there is provided a kit for determining effects of a NA editing procedure. The kit includes a set of a plurality of primer molecule types {PRₜ} designed to provide amplification of expected editing sites {λₘ} of the NA editing procedure, whereby the expected editing sites {λₘ} include at least one on-target site λ₁ and one or more off-target sites {λₘ}2, where λₘ represents an off-target or on-target site indexed m; and wherein said set of the plurality of primer molecule types {PRₜ} comprises pairs (PRM⁺, PRM⁻) of forward PRM⁺ and reverse PRM⁻ primer molecule types (PRM⁺, PRM⁻)∈{PRₜ} suitable for amplification of said on-target and off-target sites {λₘ} such that each respective forward and revers primer molecule, PRM⁺ and PRM⁻, include at least one of a forward and revers adapters. The kit is characterized in that said plurality of primer types {PRₜ} include:
forward primer molecules PRM^{+A+} including forward adapters;
forward primer molecules PRM^{+A-} including revers adapters;
reverse primer molecules PRM^{-A+} including forward adapters; and
reverse primer molecules PRM^{-A-} including revers adapters.

The kit thereby enables sequencing of all possible translocation species between at least one pair of the editing sites {λₘ}₁^{M}.

In some embodiments the primer molecule types {PRₜ} include pairs (PRM⁺ₘ, PRM⁻ₘ) of forward PRM⁺ₘ and reverse PRM⁻ₘ primer molecule types, per each site λₘ of the one or more sites {λₘ} that are to be amplified; and wherein the forward binding primer sequence PRS⁺ₘ of the forward primer PRM⁺ₘ of the site m includes an NA sequence complementary to the site's λₘ prefix sequence and the revers binding primer sequence PRS−ₘ of the revers primer PRM⁻ₘ of the site m includes an NA sequence complementary to the site's λₘ suffix.

For example, the Kit may be configured for use in a 1^{st} step, PCR1, of a Two-Step-Multiplex-Amplification process. The forwards and reverse adapters are forwards and reverse amplification adapters facilitating that all said translocation species will be amplified in said 2^{nd} step, PCR2, of the Two-Step-Multiplex-Amplification process, to produce amplicons thereof which have forwards and revers sequencing adapters from either side of the amplicon.

In another example, the Kit may be configured for use in One-Step-Multiplex Amplification process. The forwards and reverse adapters may be in this case forwards and reverse sequencing adapters.

Alternatively, or additionally, in some embodiments the Kit may be configured for use in a 2^{nd} step, PCR2, of a Two-Step-Multiplex-Amplification process. The primer molecule types {PRₜ} of the kit may include pairs (PRM⁺, PRM⁻) of forward PRM⁺ and reverse PRM⁻ primer molecule types comprising respective forward PRS⁺ and reverse PRS− binding primer sequences complementary to respective forwards and reverse amplification adapters of a 1^{st} step, PCR1, of the Two-Step-Multiplex-Amplification process. The forwards and reverse adapters of the forward PRM⁺ and reverse PRM⁻ primer molecule types may be in this case forwards and reverse sequencing adapters.

In some implementations the Kit may also include the system according to any embodiment of the present invention as described above and in more details below.

Thus, the technique of the present invention as described above and as will be described in more details below, provides for accurate detection, characterization, and quantification of off-target genome/NA-editing activity, such as indels and translocations, in pre-identified potential off-target and on-target sites, while enabling the use of multiplex amplification (multiplex PCR) followed by sequencing such as NGS, to achieve the same.

The potential off-target sites may be for example be pre-identified by unbiased discovery approaches such as GUIDE-seq⁸ or by in silico-based strategies that are homology dependent and computationally nominate potential off-target sites based on mismatch and gaps (termed “the editing distance”) between the gRNA spacer and sites in the genome of interest.

The technique of the present invention is advantageous for detecting the off-target activity with low false negative (FN) and false positive (FP) detection rates, and was proved specifically effective for detection of off-target activity at challenging loci associated with low editing rates by the genome/NA-editing procedure. Furthermore, as will be appreciated from the description below, the technique of the present invention enables the detection of alternative cut-position at off-target loci. Additionally, the technique of the present invention facilitates the use of multiplex PCR/amplification and NGS data to detect and quantify, with high sensitivity, adverse structural variations, and translocation events occurring in NA editing procedures.

Some implementations the technique of the present invention use tunable parameters that can be specified to balance between FP and FN. Accordingly the system and method of the present invention may be adjusted to follow a conservative approach, where most of the Tx reads classified as edits, count as edit events, to thereby reduce the FN rate in potential off-target loci.

Another important feature of the present invention is that it allows to infer editing rates without the need to accurately identify the NA editing procedure's (CRISPR) cut-site. This thereby facilitates identification of alternative cut-sites compared to the expected cut-site at any candidate locus. This is achieved by using a wide quantification window (e.g. in the range of 10 bases or even larger than that for each side of the DNA cut-site (e.g. instead of a narrow quantification window such as of sizes of 1-5 bases for each side of the DNA cut-site) centered at the predicted cut-site, which are may be used to avoid measuring errors resulting from PCR and sequencing). The technique of the present invention overcomes the need to use a narrow quantification window, by incorporating different prior probabilities in a larger window for each position in the reference sequence, thereby providing a more robust technique of edit events quantification.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a flow chart showing the principles of operation of a method 100 for determining effects of Nucleic Acid (NA) editing procedure 20 according to an embodiment of the present invention;

FIG. 1B shows a block diagram and a flow chart illustrating schematically a system 1000 for determining effects of Nucleic Acid (NA) editing procedure 20 according to an embodiment of the present invention, and an embodiment of a method 100.1 and how it may be implemented by the system 1000 for achieving the same;

FIG. 1C is a self-explanatory illustration depicting the categorization and classification of adverse effects in the scope of certain embodiments of the present invention;

FIG. 1D is a flowchart of a method 100.2 to statistically determine actual INDEL activity of an NA edit Procedure according to an embodiment of the present invention;

FIG. 1E is a flowchart of a method 100.3 to statistically determine actual TRANSLOCATION activity of an NA edit Procedure according to an embodiment of the present invention;

FIG. 1F is a schematic self-explanatory illustration of all four possible translocation species depicted for example for chromosomes 3 and 10 p arms;

FIG. 2A illustrates conventional PCR amplification techniques which are not adapted for producing amplicons suitable for sequencing for certain translocation species FIGS. 2B and 2C schematically illustrates a multiplex amplification according to embodiments of the present invention capable of producing amplicons suitable for sequencing of all four translocation species; in which FIG. 2B illustrates a primer kit according to the present invention for use in multiplex amplification to yield the four translocation species; and FIG. 2C schematically illustrates a multiplex amplification (which may be carried out in either a one-step or two-step multiplex amplification) with a primer kit according to the present invention for producing translocation amplicons that facilitate the sequencing of four translocation species;

FIGS. 3A and 3B are block diagrams illustrating two respective kits 410 and 420 for determining adverse effects of an NA editing procedure according to two embodiments of the present invention;

FIG. 4 is a self-explanatory illustration depicting tests conducted for detecting and quantifying off-target activity according to some embodiments of the present invention.

### DETAILED DESCRIPTION OF EMBODIMENTS

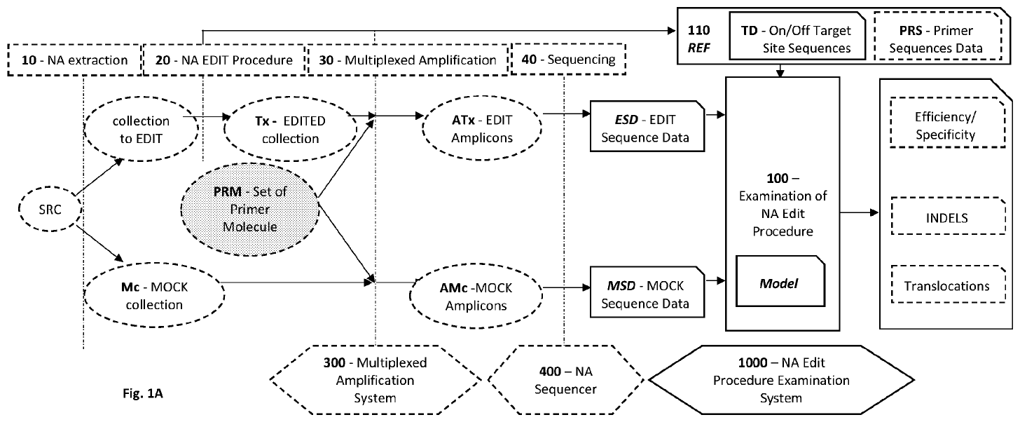

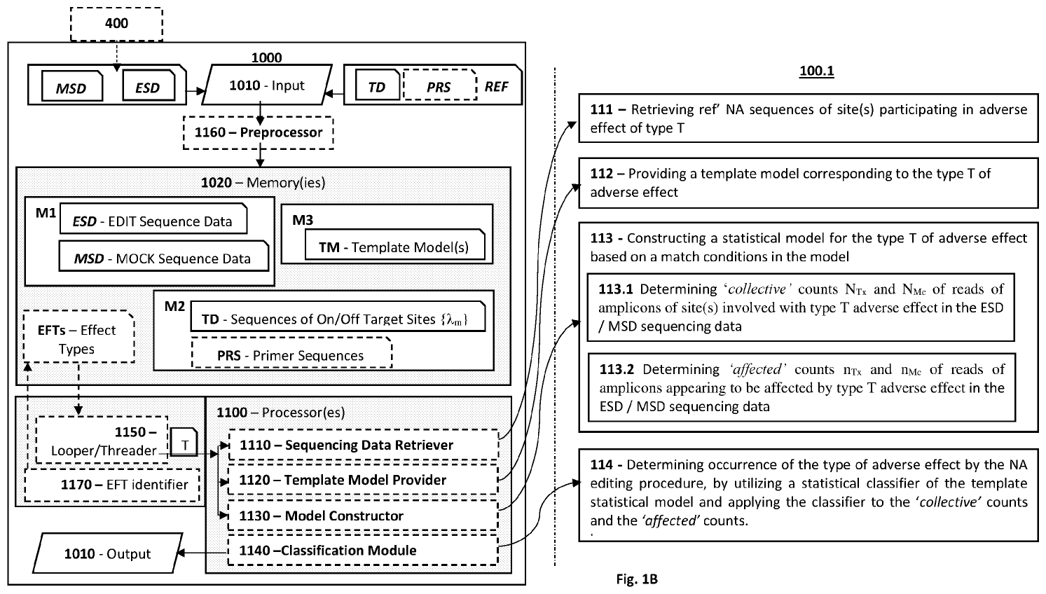

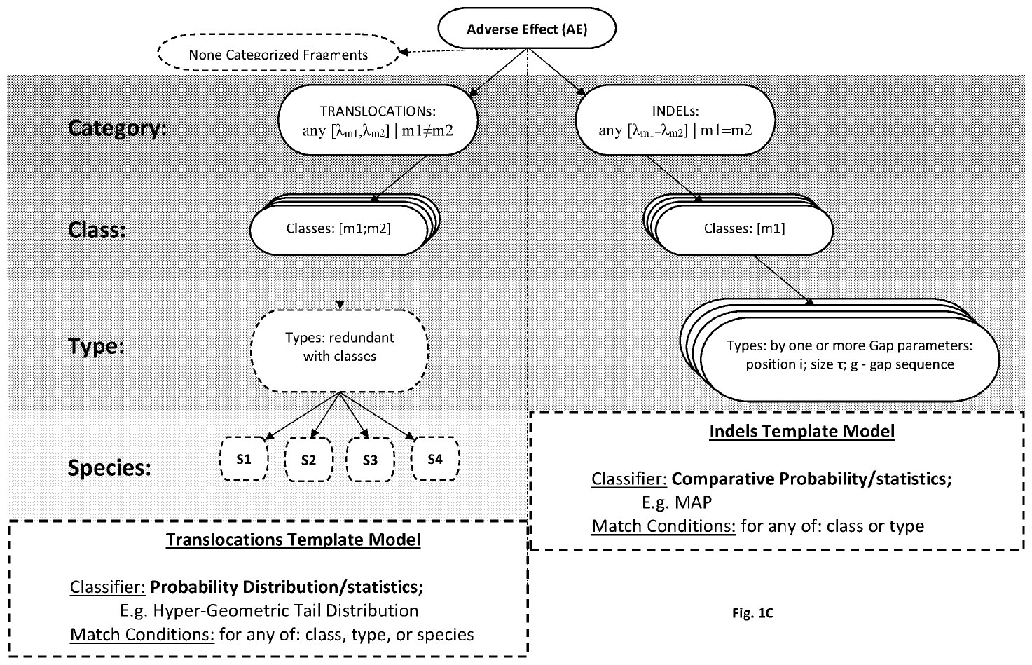

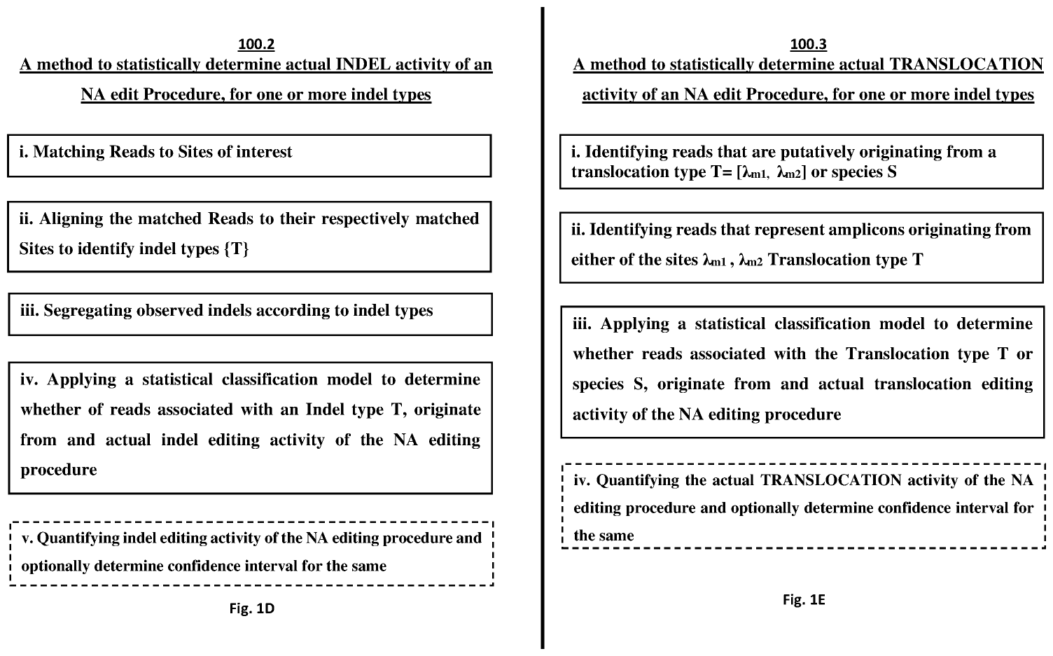

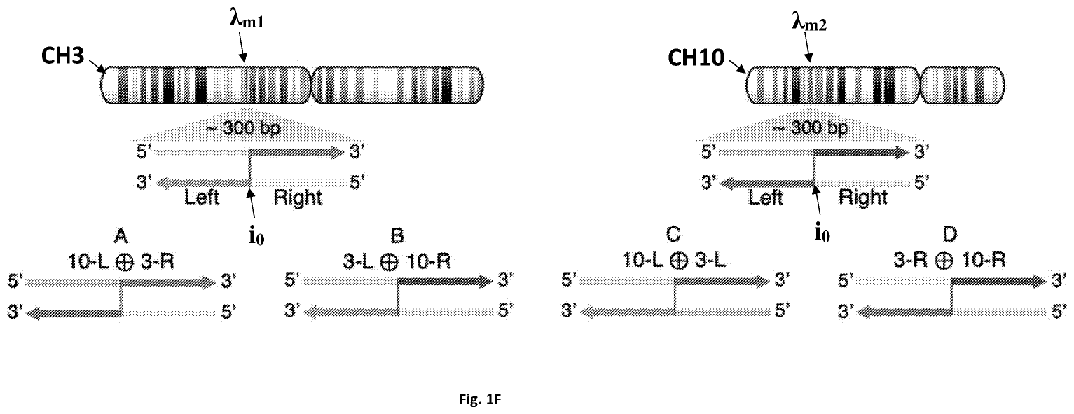

Reference is made together to FIGS. 1A to 1F, in which some embodiments of the system and methods of the present invention are schematically illustrated. It would be appreciated that like reference numerals in the figures described below designate the similar underlying entities (system modules/utilities, method operations or other objects) relating to the present invention. Optional features (such as system elements/modules and/or method operations/steps) are marked with dashed lines in the figures.

FIG. 1A is a flow chart showing the principles of operation of a method 100 for determining effects of Nucleic Acid (NA) editing procedure 20 according to an embodiment of the present invention.

The optional operations 10 to 40 described herein the following are preliminary operations which are not necessarily included or implemented by the method/system 100/1000 of the present invention.

The optional preliminary operations 10 and 20 include provision of collections a first collection Tx and second collection Mc of NA sequences originated from the same NA source SRC, whereby the first collection Tx is an edited collection of NA sequences from the NA source SRC, to which a certain NA editing procedure 20 is applied, and the second collection Mc is a control (mock) collection of NA sequences of the NA source, to the NA editing procedure 20 is not applied. Preliminary operation 10 designates the extraction of NA sequences of the collections Tx and Mc from the NA source SRC, and operation 20 designates the NA editing procedure applied to the NA sequences of the collection Tx. The FIG. 1A exemplifies a case where the extraction of the first/edited collection Tx precedes the NA editing procedure 20. It would be however appreciated by those verse in the art that this is only a none-limiting example and that the operation 10 for the first/edited collection Tx may not necessarily precede the NA editing procedure 20, as in some cases the NA editing procedure 20 may be applied directly to the source SRC or part thereof, and the extraction 10 first/edited collection Tx from the source SRC may thus be followed after the editing procedure.

The optional preliminary operation 30 includes applying amplifications to the edited Tx and control Mc collections respectively, to obtain respective amplified products/amplicons ATx and AMc corresponding to said edited Tx and control Mc collections respectively.

Preferably, the amplifications of operation 30 are conducted by applying multiplexed amplifications 30 with a plurality of primer types {PRₜ} selected to simultaneously amplify a plurality of desired sites of the NA sequences of the edited and control collections Tx and Mc. As will be appreciated by those versed in the art, provided that a technique for determining effects of Nucleic Acid (NA) editing procedure 20 is capable of handling the sequencing results of multiplexed amplification (which is the case for the technique of the present invention), the multiplexed amplification (e.g. such as multiplex PCR) is advantageous for this purpose over a singleplex amplification (e.g. as singleplex PCR) since multiplexed amplification provides for simultaneous amplification of multiple different NA sites of interest, thus enabling to efficiently amplify simultaneously different on target and of target sites of the NA editing in relatively short time.

In this regard, as would be readily appreciated by those versed in the art, after knowing the present invention, that for at least the purpose of detection of INDEL adverse effects of NA editing the technique of the present invention may also be carried out based on the singleplexed amplification. However in such embodiments employment of multiple singleplexed might be required, each per each NA site at which INDEL adverse effects of NA editing might be expected. In other words for observing INDELS in each of the M number of expected on-target and off-target sites of the NA editing procedure, in the order of M singleplexed amplification processes will be resequenced, thus yielding long and cumbersome amplification process (which may be dismissed/replaced by a single multiplex amplification).

Another advantage of the use of the multiplexed amplification over a singleplex amplification, as contemplated by the inventors of the present invention, is that such multiplexed amplification facilitates the amplification of NA sequences presenting TRANSLOCATIONs between the on-target and/or the off-target sites of the NA editing.

In this regard, as would be readily appreciated by those versed in the art, after knowing the present invention, use of singleplexed amplifications for at least the purpose of detection of TRANSLOCATION adverse effects of NA editing, is generally not practical. This is because each type of TRANSLOCATION adverse effects involves a combination at least two NA sites (e.g. two sides of the expected on-target and/or the off-target sites of the NA editing procedure). The number of such possible combination is thus in the order of M² where M presents the number of the expected on-target and off-target sites of activity of the NA editing procedure. This (M²) is typically a too large number of singleplex multiplications for being practically conducted. On the other hand, the inventors of the present invention have understood that using a multiplexed amplification of the on-target and off-target sites, will inherently amplify also at least some of the translocations between said sites. This makes the use of multiplexed amplification particularly advantageous for observation/detection of TRANSLOCATION adverse effects.

For clarity, in the following description the term multiplexed-amplification is used to describe the type of NA amplification used by the technique of the present invention. However, in view of the above explanations, it should be appreciated that the present invention is not necessarily limited to multiplex amplification, and instead a singleplex amplification may be conducted without departing from the scope of the present invention, particularly when INDEL activity of the NA editing procedure is to be assessed in only one or few sites.

Preferably, for improve accuracy and reliability of the technique of the present invention, the multiplexed amplifications 30 of the edited and control collections Tx and Mc are conducted with similar primer types. The multiplexed amplifications 30 may be conducted utilizing any known or future multiplexed NA amplification techniques, some known none-limiting examples therefore are multiplexed NA amplification techniques that are based on multiplex Polymerase Chain Reaction (PCR). It would be appreciated however that the technique of the present invention is not limited to this specific type of NA amplification and other amplification or enrichment techniques might be used. It might also be appreciated that when the NA Editing effects on a single stranded NA sequences, such as RNA, are to be examined by the technique of the present invention, (i.e. in embodiments where the edited and control collections Tx and Mc are collections of single stranded NAs), a preliminary step of the NA amplification ay include transcription/conversion of the single stranded NAs of the edited and control collections Tx and Mc to corresponding double stranded NAs (such as DNAs), and this step may then be followed by double stranded amplification, such as multiplexed PCR.

In this regards, as generally known in the art, an NA editing technique which is directed to edit a certain one or more on-target site in the NA sequences, e.g. indicated here λ1, may potentially affect/edit one or more potential off-target sites, e.g. indicated here {λₘ}₂^{M} in the NA sequences. This may be for example due to possible complemental similarity of base sequences of the off-target sites to the base sequence of the guide RNA used in the NA editing procedure, which may lead the guide RNA to bind to the off-target sites. Preliminary target information TD about expected editing sites {λₘ}₁^{M} of the NA editing procedure (M designating a number of the expected on-target and off-target sites indexed m) may for example be assessed based, for instance, on the unbiased genome-wide approaches or by other technique as for example mentioned above.

Accordingly, for the purpose of the present invention, the applying multiplexed amplifications 30, that are applied to the edited Tx and control Mc collections respectively, are preferably designed such some, or more preferably all, of the expected editing sites {λₘ}₁^{M} of the NA editing procedure, of both the edited Tx and control Mc collections, would be amplified thereby. Accordingly, the amplified products/amplicons ATx and AMc corresponding to the edited Tx and control Mc collections respectively, include amplicons of some, and preferably all, of the expected editing sites {λₘ}₁^{M} of the edited Tx and control Mc collections respectively.

For example, this may be achieved with present NA amplification techniques, such as multiplexed PCR, by conducting the NA amplifications operations 30 using a selected set of a plurality of primer molecule types {PRₜ} that are suitable for amplification of said on-target and off-target sites {λₘ}₁^{M} in the edited and control NA collections. The primer molecules of the selected set {PRₜ} may be selected according to the target information TD about the expected editing sites {λₘ}₁^{M} of the NA editing procedure. The primer molecules of the selected set {PRₜ} may for instance include, or be constitutes of, matched pairs (PRM⁺ₘ, PRM⁻ₘ) of ∈{PRₜ} that respectively match to certain prefix-primer-sequence PRS⁺ₘ and suffix-primer-sequence PRS⁻ₘ primer molecule types {(PRM⁺ₘ, PRM⁻ₘ)}₁^{M} of each or at least some of the expected editing sites {λₘ}₁^{M}.

The optional preliminary operation 40 includes sequencing of the multiplexed amplifications products/amplicons ATx and AMc of the edited and control collections, Tx and Mc, by which sequencing data, ESD and MSD, including respective pluralities of reads, R_{Tx}={rᵢ^{Tx}} and R_{Mc}={rⱼ^{Mc}}, of the multiplexed amplifications' products/amplicons ATx and AMc of each of the edited and control collections, is obtained. In this regard it should be understood that the sequencing operation 40 is not limited to any particular sequencing technique or sequencer system 400 and may be conducted with any suitable existing or future NA sequencing technique sequencer system 400, as known or will be known in the art.

As indicated above, the operations 10 to 40, are optional preliminary operations which may be performed in the scope of the method 100 of the present invention, or prior thereto. According to the method 100 of the present invention, the sequencing data ESD and MSD including the respective pluralities of reads, R_{Tx}={rᵢ^{Tx}} and R_{Mc}={rⱼ^{Mc}}, of the amplicons ATx and AMc is received and processed for constructing, per each particular type of adverse effect of one or more types of possible adverse effects of said NA editing procedure (not necessarily for all possible adverse effects), a statistical model of occurrence of that type of adverse effect by the NA editing procedure. The statistical model generally employs a classifier to determine whether an adverse effect of that type occurs due-to/by the NA editing procedure, or otherwise the that type of adverts effect is observed due to errors/artifacts (generally referred to herein as NOISE), caused by either the Amplification or the Sequencing preliminary operations 30 and/or 40.

Accordingly, in the technique of the present invention the classifier of the statistical model, statistically determines a likelihood that the observed type of adverse effect is a type of adverse effect occurring due to the NA editing procedure based on a comparison between numbers of observed amplicons with NA sequences corresponding to that type of adverse effect of in each of the ATx and AMc amplicons of the edited and control/mock collections respectively.

As a result of this process, accurate estimation of the types of adverse effects obtained by the examined NA editing procedure is obtained. Data indicative of the assessment/quantification of the occurrence of the type(s) of adverse effect by the NA editing procedure, of one or more categories (translocations or indels), is then output, to enable determination of suitability safety of application/use of NA editing procedure (e.g. on the NA sequences of the source SRC). Alternatively or additionally, data about the efficiency/specificity of the NA editing procedure may also be determined and output, based on the identified types of adverse effects.

Further details and examples of the technique, system 1000, and method 100 according to an embodiment of the present invention, will now be described with reference to FIG. 1B. FIG. 1B is block diagram illustrating the system 1000 for determining data indicative of the effects of Nucleic Acid (NA) editing procedure according to an embodiment of the present invention.

The system 1000 may be used for determining and outputting data indicative of the effects of Nucleic Acid (NA) editing procedure, The system 1000 includes:
an input 1010 adapted to receive sequencing data ESD and MSD associated with the sequencing of the edit and control amplicons ATx and AMc, as well as optionally reference data REF including reference target data TD indicative of the sequences of the on-target and off-target sites of the NA edit procedure 20, and optionally primer sequences data PRS indicative of the primer sequences (e.g. prefix and suffix) of the on-target and off-target sites (these may or may not be similar to the sequences of the primer molecules used for the amplification 30);
one or more memory modules 1020 capable of storing the sequencing data ESD and MSD, the reference data REF, template model(s) TM for use in the classification, and possibly a list EFT of one or more types {T} or classes to be examined; and
one or more processing modules 1100 configured and operable for carrying out the method 100 according to one or more embodiments of the present invention, for processing the sequencing data ESD and MSD based on the reference data, and the suitable template model and thereby determine whether the at least one type T of adverse occurs by the NA edit procedure 20; and
an output adapted for outputting data indicative of the probability of occurrence of the at least one type T of adverse effect by the NA edit procedure 20. The output data being indicative of whether to sue/administer the NA edit procedure 20 for a particular use on a subject/object having a collection NA sequences similar to the source SRC. The output may acquire any of various forms, for example by: output signals receivable/readable by another system and/or by visual display or presentation and/or by audio notification/signal and/or by recordation of data on an output memory section or data storage.

More specifically the input 1010 is adapted to receive sequencing data indicative of respective the read results ESD and MSD of the edit and control amplicons ATx and AMc. This includes pluralities of reads, R_{Tx}={rᵢ^{Tx}} and R_{Mc}={rⱼ^{Mc}} [CAN YOU POVIDE THE ORDER OF NUMBER OREADS] obtained by sequencing of multiplexed amplifications products/amplicons ATx and AMc of edit and control/mock collections of NA sequences.

The memory 1020 or a section M1 thereof, stores the respective pluralities of reads, R_{Tx}={rᵢ^{Tx}} and R_{Mc}={rⱼ^{Mc}}, of the sequencing data ESD and MSD. The memory 1020 or a section M2 thereof, also stores the reference data REF which is indicative of at least one reference NA sequence of at least one respective site λₘ of the expected editing sites {λₘ}₁^{M} of the NA editing procedure. The memory 1020 or a section M2 thereof, may also store sequences data PRS indicative of the primer sequences (e.g. prefix and suffix) of on-target and/or off-target sites. The primer sequences data PRS may be for example indicative of the set of the plurality of primer molecule types {PRₜ} that are used in the multiplexed amplifications 30 of the expected editing sites {λₘ}₁^{M} of the NA editing procedure 20.

It should be noted that according to some embodiments of the present invention system 1000 may optionally also include (or be connected to (e.g. directly)) of the sequencing utility 400 which is capable of sequencing of the multiplexed amplifications products/amplicons of the first and second collections of NA sequences. In such embodiments the input 1010 may be connectable to the sequencing utility 400 for receiving the sequencing data therefrom.

It should be noted that according to some embodiments of the present invention system 1000 may optionally also include a preprocessor 1160 adapted to apply one or more preprocessing operations to the received sequencing data ESD and MSD (although not specifically depicted in the figures, these preprocessing operations may be part of the operations of the method 100). The preprocessing may for instance include adjusting the reads, R_{Tx}={rᵢ^{Tx}} and R_{Mc}={rⱼ^{Mc}} of the sequencing data ESD and MSD by carrying out at least one of the following:
trimming of sequencing adapters from the reads;
merging pair-end reads; and
filtering out low-quality reads.

The memory 1020, or a section thereof M3, may also store at least one template statistical model TM corresponding to at least one category of adverse effects, indels and/or translocations. The template statistical model TM may for example include a set of match conditions and a classifier (e.g. matrix). The match conditions are designated to be applied for comparison between the sequencing data ESD and MSD and a part of the reference data REF that corresponds to the type T of effect looked for, to determine properties of the sequencing data (e.g. the collective and affected counts) indicative of occurrence of this effect T. The classifier is designated to process the properties of the sequencing data determined by the match conditions, to determine/assess occurrence of that adverse effect T due to the NA editing procedure 20.

It should be noted that the list EFT of one or more types {T} or classes of adverse effects which are to be examined by the system 1000 may be received as input, or may be internally identified, e.g. based on the category or class of adverse effects to be processed by the system.

In the latter case the system may include an ETF identifier module 1170 capable of processing the reference data (e.g. the sequences of the on and off targets sites, and possibly also the sequencing data to identify a list of the possible adverse effects to be processed by the system. For instance, in embodiments directed to translocation detection, identifier module 1170 may include in the list EFT of types/classes {T} of adverse effects all the pair combinations of different on and off targets sites [λm1,λm2]. Alternatively or additionally, in embodiments directed to indel detection, per each class/site [λm] in which types of indels are to be detected, the identifier module 1170 may process the sequencing data ESD and MSD after being aligned to the site λm of the class [λm] (see alignment discussion below), to identify gaps in the aligned sequences and process/identify the types T of the indels to be included in the list based on the relevant properties of the gaps (e.g. their i position within the respective site λm; their sizes for example in terms of number of bases—these may be positive of insertion-indels and negative for deletion-indels; or the sequence of bases introduced therein, in case of insertions indels). Accordingly the identifier module 1170 may list EFT the types/classes {T} adverse effects that should be processed by the system 1000.

In some implementations the system also includes a looper/threader module which is configured and operable to process the EFT the types/classes {T} adverse effects that should be processed by the system 1000 and operate the processor, for instance sequentially in a loop, and/or in parallel (e.g. by multithreaded operation), to process the sequencing data ESD and MSD per each type T of adverse effect in the list EFT to determine the likelihood of occurrence for each said type T.

In turn, type T of adverse effect the processor 1100 is configured and operable for processing the sequencing data ESD and MSD according to the method 100 of the present invention, based on the reference data REF and the template statistical model TM, for applying the template statistical model TM suitable to the type T, to sequencing data ESD and MSD, and to thereby assess/determine a the occurrence of at least one type T of adverse effect by the NA editing procedure.

FIG. 1B illustrated for example an embodiment 100.1 of the method 100 of the present invention which may be implemented by the processor 1100, and also illustrates optional processing modules 1110 to 1140 of the processor 1100 which are configured and operable to implement the respective operations of 111 to 114 of the embodiment method 100.1. To this end the processor 1100 may be adapted to process the sequencing data for the/each type T of adverse effect by carrying out the following:
a. The Sequencing Data Retriever module 1110 may be adapted to carry out operation 111 of the embodiment method 100.1 for retrieving from the reference data REF stored in the memory 1020, reference NA sequences of one or two sites [λₘ₁,λₘ₂] that participate in that type T of adverse effect;
b. The Template Model Provider 1120 may be adapted to carry out operation 112 of the embodiment method 100.1 for obtaining from the memory 1020 the template statistical model TM corresponding to the category of the type of adverse effect;
c. The Model Constructor 1130 may be adapted to carry out operation 113 of the embodiment method 100.1 for constructing a statistical model for the type T of adverse effect based on the template statistical model TM and the reference data REF. This may be implemented by carrying out the following:
   Operation 113.1 includes processing the sequencing data ESD and MSD of the amplified edit and mock collections to determine or assess respective ‘collective’ counts N_{Tx} and N_{Mc} of reads of amplicons, which are associated with the one or two sites [λₘ₁,λₘ₂] participating in the particular type T of adverse effect. The processing may be carried out by matching these amplicons to the reference NA sequences of the one or two sites [λₘ₁,λₘ₂] according to a ‘collective’ count match condition which is designated by the template statistical model TM. Then respectively counting the reads of amplicons of each of the edit and mock collections that satisfy the ‘collective’ count match condition, to yield a determination or assessment of the respective ‘collective’ counts N_{Tx} and N_{Mc};
   Operation 113.2 includes processing the sequencing data ESD and MSD of the amplified edit and mock collections to determine or assess respective ‘affected’ counts n_{Tx} and n_{Mc} of reads of allegedly affected amplicons in which the particular type of adverse effect T is observed. The processing may be carried out by matching these amplicons to the reference NA sequences of the one or two sites [λₘ₁,λₘ₂] according to an ‘affected’ count match condition designated by the template statistical model TM, and respectively counting the reads of amplicons of each of the edit and mock collections that satisfy the ‘affected’ count match condition, to yield a determination or assessment of the respective ‘affected’ counts n_{Tx} and n_{Mc};
d. The Classification module 1140 may be adapted to carry out operation 114 of the embodiment method 100.1 for determining whether the particular type T of adverse effect occurs or is likely to occur, by the NA editing procedure 20. This may be achieved by applying the statistical model to the sequencing data, by carrying out the following:
   utilizing a statistical classifier of the template statistical model; and
   determining the occurrence, or computing the likelihood of occurrence, of the particular type T of adverse effect by the NA editing procedure 20. according to this statistical classifier based on the ‘collective’ counts N_{Tx} and N_{Mc} and said ‘affected’ counts n_{Tx} and n_{Mc}.

As indicated above the systems and methods according to various embodiments of the present invention may be configured and/or operable for determining the occurrence adverse effects of one or both of the following categories, by the NA edit procedure:
Category 1 (INDELs): adverse effects involving one site [λₘ₁₌ₘ₂] where m1=m2; and
Category 2 (TRANSLOCATIONs): adverse effects involving two sites [λₘ₁,λₘ₂] where m1≠m2.

FIG. 1C is a self-explanatory illustration depicting the categorization and classification of these adverse effects conducted in the scope of the present invention, as well as possibly typification, and division to species certain adverse effect categories. FIG. 1C also depicts in a self-explanatory manner to examples of template statistical models suitable for determination if adverse effects of translocation and/or indel categories occur, or are likely to occur, due to the NA edit procedure 20. It would be appreciated that the statistical models provided herein present specific non limiting examples of statistical models, contemplated and tested by the inventors of the present invention to be suitable for assessing the probabilities of occurrence of adverse effects of translocation and/or indel categories. The scope of the present invention need not be essentially limited by these example models, as other statistical models may be used.

With reference to FIGS. 1A to 1C, it should be noted that in some implementations preprocessing operations (e.g. performed by the preprocessor 1160) may include preprocessing of the reads R_{Tx}={rᵢ^{Tx}} and R_{Mc}={rⱼ^{Mc}} of the sequencing data ESD and MSD for initial categorization and classification thereof to the classes of at least one of the categories depicted in FIG. 1C. This may optionally be performed after the one or more optional preliminary preprocessing steps, which may be carried out on the reads R_{Tx}={rᵢ^{Tx}} and R_{Mc}={rⱼ^{Mc}} for trimming the reads and/or merging pair-end reads, and/or filtering out low-quality reads. The initial categorization and classification preprocessing operations may be carried out as follows:
A. Assigning each read of a plurality of the reads R_{Tx}={rᵢ^{Tx}} and R_{Mc}={rⱼ^{Mc}} of the edit and mock sequencing data ESD and MSD, to a particular reference sequence, or locus of interest λₘ₁ In the reference data. This assignment may be performed by matching for each one of the read's prefix Pfx(r) or suffix Sfx(r), to one of the respective one of the prefix/forward or suffix/reverse of the primer sequences PRS of the different in the reference data. The matching of the read may be for example based on a lowest edit distance condition between the matched primer sequences PRS. Reads that do not match any of the prefix/forward or suffix/reverse of the primer sequences PRS of the different On/Off Target Sites {λₘ} may be considered as not relevant NA fragments. By the end of this operation, each, which is not considered as none-relevant NA fragment, is assigned to one of the On/Off Target Sites {λₘ} by the matching of the read's prefix Pfx(r) or suffix Sfx(r) to the respective prefix/forward primer sequence or suffix/reverse primer sequence of the site of interest λₘ₁.
B. Then a categorization of assigned the reads to at least one of the indel ant translocation categories may be conducted, by carrying out one or both of the following:
   a. Categorizing reads associated with INDELs category includes matching of the other one of the read's prefix Pfx(r) or suffix Sfx(r) to the respective other one of the prefix/forward primer sequence or suffix/reverse primer sequence of the same site of interest λₘ₁ to which the read is assigned (m1=m2). Upon determining a match (e.g. based on the lowest edit distance condition which may be similar as above), the read is categorized as of INDEL category (it is not necessarily an indel in case no insertion deletion is presented in the read). Note that the matching here may include aligning the other one of the read's prefix Pfx(r) or suffix Sfx(r) the read's prefix/suffix to the other the respective one primer of the prefix/forward primer sequence or suffix/reverse primer sequence of the same site of interest λₘ₁.
   b. Categorizing reads associated with TRANSLOCATIONs category includes matching of the other one of the read's prefix Pfx(r) or suffix Sfx(r) to the a prefix/forward or suffix/reverse primer sequence of the another site λₘ₂ of the On/Off Target Sites {λₘ} which is different than the first site λₘ₁ to which the read is assigned (m1≠m2). Upon determining a match (e.g. based on the lowest edit distance condition which may be similar as above), the read is categorized as of TRANSLOCATION category. Note that the matching here may include matching the other one of the read's prefix Pfx(r) or suffix Sfx(r) to any of the prefix/forward or suffix/reverse primer sequences of the On/Off Target Sites {λₘ} other than λₘ₁.

Note that in embodiments where only translocations are of interest, only the operation b. is performed and in embodiments where only indels are of interest, only the operation a. is performed.

At the end of the operations a. or b. each categorized read is classified according to its class. Namely for indels each categorized read rᵢ are assigned with a corresponding class: r_{i->}[λm1]=[λm1=λm2]|m1=m2; and for translocations, each relevant read rᵢ, is assigned with a corresponding class: r_{i->}[λm1,λm2]|m1≠m2. Reads not matching to the one or both categories of interest may optionally be ignored in the further processing (e.g considered as none-relevant to the category of interest or as fragments/artifacts.

In the following description, examples of template (statistical) models for determining occurrence of various respective indel types or translocation types/classes will be exemplified and described in more detail. It should be understood that the below models are provided as none limiting examples and as will be appreciated by those versed in the art after knowing the invention, other suitable models may be devised without departing from the scope of the present invention as defined in the claims. Thus, in the following at least one of modeling methods I. and II. for indel and translocations categories respectively may be performed:

I. Statistical Model for Determining INDEL Activity of the NA Edit Procedure

Reference is made to FIG. 1D, in which a flowchart of a method 100.2 to statistically determine actual NHEJ/INDEL activity of an NA edit Procedure 20, for one or more indel types (e.g. at a given locus λ), according to an embodiment of the present invention, is schematically presented. The method 100.2 may be performed by the processors 1100 of the system 1000 described above. As will be appreciated by those versed in the art the operations i to v. of the method 100.2 may be complemented by any one or more of the operations of the methods 100 and/or 100.1 described above and/or by the operations of method 100.3 described below. The method 100.2, may be performed as follows:

i. Matching Reads to Site

For indels the combinations of the preprocessing operations A. and B.a. above may be more specifically described as follows for a given site/locus of interest λₘ₁ of which indels are to be assessed:
a. providing reference data indicative of at least one reference NA sequence of the at least one respective site/locus of interest λₘ₁ for which INDEL activity of said NA editing procedure is to be assessed;
b. utilizing a ‘collective’ count match condition of said template statistical model, to identify matched reads of the multiplexed amplifications products/amplicons that match the reference NA sequence of the site/locus of interest λₘ₁ in the reference data; and

This results with obtaining, for the site of interest, λₘ₁, respective collections L_{Tx}(λₘ₁) and L_{M}(λₘ₁) of matched reads, obtained from the sequencing data ESD and MSD of the edited and control collections respectively.

In this connection it is noted that the ‘collective’ count match condition of the template statistical model of the INDEL Category 1 presents the combination of the matching conditions described above with reference to the preprocessing operations A. and B.a. This collective’ count match condition is satisfied for a read rᵢ in case the prefix and suffix regions of the read rᵢ match prefix PRS⁺ₘ and suffix PRS⁻ₘ primer sequences (PRS⁺ₘ₁, PRS⁻ₘ₁) of a respective site of interest λₘ₁.

As will be described in more details below, the sizes of respective collections L_{Tx}(λₘ₁) and L_{M}(λₘ₁) present the ‘collective’ counts N_{Tx}(λₘ₁) and N_{Mc}(λₘ₁) of reads of amplicons which are associated with Indel at least one class of adverse effects involving the site λₘ₁ as observed in the sequencing data of the corresponding edited and control collections ESD and MSD.

ii. Aligning the Matched Reads to their Respectively Matched Site to Identify Indel Types {T}

After obtaining the respective collections L_{Tx}(λₘ₁) and L_{M}(λₘ₁) of reads for each site of interest λₘ₁ (typically conducted for all of the on and off target sites), the lists/collections L_{Tx}(λₘ) and L_{M}(λₘ₁) of each site of interest λₘ₁ may be processed to identify indel types {T} appearing therein. This includes carrying out the following:
(a) aligning the matched reads in the collections L_{Tx}(λₘ) and L_{M}(λₘ₁) to the reference NA sequence of the site of interest λₘ₁. To align the reads to the locus from which they originated, an optimized version of the Needleman-Wunch algorithm³⁷ may be for example used or any other suitable alignment procedure (see for example the alignment technique discussed in PCT patent application publication No. WO2020/123906).
(b) The gaps in the aligned matched reads are identified whereby each gap representing an indel and at least one of a position i and a length/size τ of the gap represents a type T of the indel; and

At the end of this operation, for each locus λₘ₁ of interest, a list of aligned reads coming from the mock collection Mc, denoted L_{Mc} and a list of aligned reads coming from the edited collection Tx, denoted L_{Tx}, are obtained. The comparison of these two lists may be used basis for quantifying the indel activity detected at the loci λₘ₁ of interest.

iii. Segregating Observed Indels According to Indel Types

Then, for each locus site λₘ₁, Tx and M read lists, L_{Tx}(λₘ) and L_{M}(λₘ₁), are converted into several more focused lists pertaining to specific indel types T and positions, L_{TI}(λₘ₁,T) and L_{M}(λₘ₁,T). For a given indel type T (and a given locus λₘ₁) the information may for example be summarized in the form of a table where each column represents a position i on the reference locus λₘ₁.

Thus, in order to typify the observed indels, further processing of the sequencing data of the indel reads L_{TI}(λₘ) and L_{M}(λₘ₁) may include segregating the collections L_{Tx}(λₘ₁) and L_{M}(λₘ₁) of reads matching the site of interest λₘ₁, to form respective two sub-collections L_{Tx}(λₘ₁,T) and L_{M}(λₘ₁,T) of reads per each indel type T of interest which is observed in the reads (typically all types are of interest). It should be noted that the reads may be typified according one or more of the following characteristics:
a size/length τ of bases introduced-to or deleted-from the matched read relative to the reference NA sequence of the site/locus of interest λₘ₁, and
a position i along the site of interest λₘ₁ at which said bases are introduced-to or deleted-from; and

The segregation/conversion to the sub-collections L_{TI}(λₘ₁,T) and L_{M}(λₘ₁,T) of reads may for example include identified gaps in the aligned matched reads with properties of a gap representing each certain type T of indel based on an ‘affected’ count match condition of the template statistical model of Indels. The ‘affected’ count match condition of the template statistical model of Indels may be a condition satisfied upon fulfillment of a predetermined set one or more of the following conditions:
the position i of an identified gap in an aligned matched read is similar to a position î of the gap in the type T of indel;
a size τ of the gap of an identified gap in an aligned matched read is similar to a size \hat{ }τ of the gap in the type T of indel;
a nucleotide base sequence in the gap of an identified gap in an aligned matched read has a degree of similarity with a nucleotide base sequence of the type T of indel (e.g. below to certain edit distance threshold).

To this end, in some embodiments all the indel reads L_{Tx}(λₘ) and L_{M}(λₘ₁) may be assign into indel types {T} (for instance per position and/or indel gap size) to obtain lists of reads L_{M}(τ,i) and L_{Tx}(τ,i), where τ denotes the indel type, e.g. insertion of length 3, and i denotes the index of the position at the given reference sequence. Note that reads that are perfectly aligned to a reference site of interest λₘ₁, are not members of any of the above lists of reads L_{M}(τ,i) and L_{Tx}(τ,i) (for off-target sites these are typically a majority as only small editing effects are expected therein). Also note that reads may represent more than one type T of indel implicated several gaps identified by the alignment. The sizes of respective sub-collections/lists L_{Tx}(λₘ₁,T) and L_{M}(λₘ₁,T) present the ‘affected’ counts n_{Tx}(λₘ₁,T) and n_{Mc}(λₘ₁,T) of reads of amplicons, in which said particular type T of adverse effect is observed at site λₘ₁.

It should be noted that in some embodiments the indel type T is characterized by only one of the size/length τ of bases of the gap or the position i of the gap in the respective site's sequence. Alternatively, in some embodiments the type T is characterized by the combination of both the size/length τ of bases and the position i.

The following description exemplifies the application of a statistical model to the ‘affected’ counts n_{Tx}(λₘ₁,T) and n_{Mc}(λₘ₁,T) and ‘collective’ counts N_{Tx}(λₘ₁) and N_{Mc}(λₘ₁) indicated above to determine whether the NA edit procedure causes an adverse effect of indel of type T at the site λₘ₁. This may be performed for each of a plurality of indel types T of each of a plurality of sites λₘ₁. For clarity, in the following description which exemplifies the statistical modeling for a certain indel of type T at a certain site λₘ₁, the indication of the parameters (λₘ₁,T) are omitted from the ‘affected’ and ‘collective’ counts n_{Tx},n_{Mc}, N_{Tx} and N_{Mc}.

iv. Applying a Statistical Classification Model to Determine Whether of Reads Associated with an Indel Type T Originate from and Actual NHEJ/Indel Editing Activity of the NA Editing Procedure

The technique of the present invention provides for distinguishing indel reads resulting from molecules edited by the NA edit procedure (edited reads) from indel reads derived from various sources of noise (e.g. from the amplification or sequencing). This distinction is based on the ability to assess whether a specific indel originates from edit event of the NA edit procedure or is a result of experimental background noise (such as sequencing artifacts or erroneous read assignments). The model used according to an embodiment of the present invention for identifying/distinguishing edited reads from noise, is based on the comparison of indel statistics in the amplicons ATx of the edited collection vs the amplicons of the control/mock collection AMc of the edited collection (e.g. utilizing comparative Probability/statistics as exemplified in FIG. 1C). A person of ordinary skill in the art would readily appreciate after knowing the present invention, that other statistical models may also be suitable for the purpose of the present invention.

For each indel type, T, the system 1000 or method 100 according to an embodiment of the present invention, applies a statistical inference classifier to classify whether indel events of type T in site λₘ₁ in the reads of amplicons ATx of the edited collection, are a result of an editing event introducing the indel of type T to the site λₘ₁ or are noise (such as amplification/PCR noise or sequencing noise which also occur in the mock sample). As indicated above, the indels types T are identified with comparison to the reference NA sequence of the respective site λₘ₁ (e.g. via the alignment operation). The type T indel events observed within the reads of the edit collection Tx, are classified as originating from an edit event or from background noise. All Tx reads with type T indels in, which are positively classified (i.e. as edit event), are considered as edited reads. This process may be repeated for all or some of a plurality of indel types T (e.g. for each pair of the identified gap's size and position (τ,i)). Eventually, all reads that are classified as positive in at least one indel type T are marked as edited reads.

Optionally, in order to improve the efficiency of the process, only indel types T whose position i along the site of interest λₘ₁ is within a predetermined a window around an expected cut-site/position i₀ of the NA editing procedure at the site of interest λₘ₁ are processed (i.e. for classification). For example the window size may be in the order of several tens of base-pairs (e.g. 20 bp) and indel types observed in other positions outside that window may be discarded. This is because (one may expect that an indel originating from an edit event will be a result of a double strand break at/near the cut-site/position i₀ of the NA editing procedure.

Accordingly, for each indel type T of interest, a classifier (is used to determine whether reads that represent this indel T, originate from an editing event, or from background noise. This is based on the above indicated the ‘affected’ and ‘collective’ counts n_{Tx},n_{Mc} N_{Tx} and N_{Mc} associated with the site λₘ₁ and the type T of the indel.

In some embodiments the template statistical model provided according to the present invention for the assessment of INDEL activity of the NA editing procedure includes a statistical maximum likelihood classifier may be used for this purpose as the statistical inference classifier.

Alternatively, or additionally in some embodiment's prior probabilities P(edit) and P(no edit) may be obtained or estimated, indicative of the probabilities of occurrence or none-occurrence of an edit event causing the respective indel type T. In such embodiments, the template statistical model provided according to the present invention for the assessment of INDEL activity of the NA editing procedure may include a more accurate statistical classifier including a Maximum A Posteriori estimator (MAP), such as a Bayesian classifier may be used to obtain a more accurate statistical classification.

In this regard it should be noted that prior probabilities (priors) P(edit) and P(no edit) are complementary Prior Probabilities indicative of the probability of edit or no-edit occurrence of the indel type T where here complementary P(edit)=1−P(no edit). The prior probabilities P(edit) and P(no edit) may be part of the model and may be provided as functions or lookup tables, which depend on a distance between a position i along the site of interest λₘ₁ at which the indel of the type T is observed and the expected cut-site position i₀ of the NA editing procedure at the site of interest λₘ₁.

The priors may be defined based on the basic principles of the NA editing procedure (e.g. based on the basic principles of CRISPR activity) or based on experimental data of the NA editing procedure. In some embodiments these priors are configurable. In some embodiments these Prior Probabilities are adjusted depending on the base-distance between the indel's position i and the cut site of the DNA editing procedure.

For example, the prior probability P(edit) may be set to a predetermined value (e.g. about 0.5) within a predetermined range for example of −6≤i≤6 when the index i corresponds to a position relative to the expected cut-site position i₀. The priors can then be P(edit,i)=exp(−α|i−i₀|) for any positive a. The priors will then be decreasing in order, according to distance |i−i₀| between the position i and the expected cut-site position. In some embodiments these priors can be 0.5, 0.1, 0.01, 0.001, 0.0001. Note, that selection/configuration pf the system and/or method of the present invention with non-zero Prior Probabilities away from the cut-site i₀ allows for the detection of alternative cut-sites in the classification window and are fully user-configurable.

Indeed, it should be understood that the maximum likelihood classifier is a private case of the Maximum A Posteriori estimator where the prior probabilities P(edit) and P(no edit) are not know or estimated. E.g. by trivially setting P(edit) and P(no edit) to the trivial values P(edit)=0.5 and P(no edit)=0.5 in the Maximum A Posteriori estimator, the statistical maximum likelihood estimator (MLE) classifier is obtained. To this end in some embodiments the prior probabilities P(edit) and P(no edit) of said MAP estimator may be set as fixed trivial probabilities independent of position P(edit)=P(no edit)=0.5, and the MAP estimator thereby functions as maximum likelihood estimator (MLE).

In the following description the application of the template statistical model for indels is described based on a specific non-limiting example in which Bayesian classifier is used as MAP estimator of the template statistical model. In this case the application of the template statistical model to the respective ‘collective’ counts N_{Tx} and N_{Mc} of reads and the respective ‘affected’ counts n_{Tx} and n_{Mc} includes computing the Bayesian classifier to determining whether the NA editing procedure has caused edit events resulting with the indels of the type T.

For example, for a given indel type T˜(τ,i) (gap size τ and a indel position i) n_{Tx}=|L_{Tx}(τ,i)| and n_{M}=|L_{M}(τ,i)|. Based on these observed numbers, the model determines whether the observation is more likely to have originated from an edit event—P(edit|n_{Tx},n_{M}), or to represent background noise—P(no edit|n_{Tx},n_{M}). MAP classifies an indel of type T as originating from an edit event when the posterior P(edit|n_{Tx},n_{M}) is higher.

To this end the MAP estimator probabilities may be compared using Bayes rule as follows:

P(edit|n_{Tx},n_{M})>P(no edit|n_{Tx},n_{M})⇔P(edit)·P(n_{Tx},n_{M}|edit)>P(no edit)·P(n_{Tx},n_{M}|no edit) (1)

wherein P(edit|n_{Tx},n_{M}) and P(no edit|n_{Tx},n_{M}) are the respective probabilities that an edit hypothesis and a no-edit hypothesis are valid given the observed ‘affected’ counts n_{Tx} and n_{Mc} of indels of type T in the in the reads of the amplicons ATx and AMc of the edit and control sequences; P(edit) and P(no edit) are the above indicated priors; P(n_{Tx},n_{M}|no edit) is a probability of observation of the ‘affected’ counts n_{Tx} and n_{Mc} in the edited and control sequences under an assumption that there was no edit causing the ‘affected’ count n_{Tx} observed in the edited sequences and P(n_{Tx},n_{M}|edit) is a probability of observation of the ‘affected’ counts n_{Tx} and n_{Mc} under an assumption that there was an edit causing the ‘affected’ count n_{Tx} observed in the edited sequences.

In some embodiment as exemplified herein the template statistical model includes utilizing hyper-geometric distribution for computing the probability P(n_{Tx},n_{M}|no edit) of observation of the ‘Affected’ counts n_{Tx} and n_{Mc} under the no edit assumption. In this regards, the hypergeometric distribution is a discrete probability distribution that describes the probability of b=n_{Tx} successes (random draws for which the object drawn has a specified indel feature) in n=N_{Tx} draws, without replacement, from a finite population of size N=N_{Tx}+N_{M} (size of both the mock and the edit sequenced populations) that contains exactly B=n_{Tx}+n_{M} objects with that indel feature, wherein each draw is either a success or a failure. This model means that all indels are equally likely to occur in a M read as in a Tx read. For the reasons explained below, using the hypergeometric distribution provides accurate statistical modeling for the probability of a scenario in which n_{Tx}+n_{M} indel events (of type T) are observed in the edit sample and the mock sample in case the events n_{Tx} in the edit sample are not a result of DNA editing.

It should be however understood that for some implementations using different distributions for modeling the probability of P(n_{Tx},n_{M}|no edit), may also be possible, for instance using the Binomial Distribution (e.g. although using such other distribution may generally be less suited for this scenario and may yield less accurate results in various circumstances).

To this end, given collective counts N_{Tx} and N_{M}, indicated above, without loss of generality (due to the symmetry of the hypergeometric distribution), the hypergeometric distribution HG(b; N, B, n) for P(n_{Tx},n_{Mc}|edit) may be defined as:

$$\begin{matrix}P ( {n}_{T x} , {n}_{M} \vert no edit ) ∼ H G ( b ; N , B , n ) = \frac{( \begin{matrix}B \\ b\end{matrix} ) ( \begin{matrix}N - B \\ n - b\end{matrix} )}{( \begin{matrix}N \\ n\end{matrix} )} \\ = \frac{( \begin{matrix}{n}_{T x} + {n}_{M c} \\ {n}_{T x}\end{matrix} ) ( \begin{matrix}{N}_{Tx} + {N}_{M c} - {n}_{Tx} - {n}_{M c} \\ {N}_{Tx} - {n}_{Tx}\end{matrix} )}{( \begin{matrix}{N}_{Tx} + {N}_{M c} \\ {N}_{Tx}\end{matrix} )}\end{matrix} \qquad ( 2 )$$

Where N=N_{Tx}+N_{Mc}, b=n_{Tx}, B=n_{Tx}+n_{Mc}, n=N_{Tx}

In some embodiment as exemplified herein the template statistical model includes utilizing the binomial distribution for computing assessing the probability P(n_{Tx},n_{M}|edit) of observation of the ‘affected’ counts n_{Tx} and n_{Mc} under the edit assumption. P(n_{Tx},n_{M}|edit) represents the probability of seeing the observed number n_{Tx} of indels in the reads of the edit amplicons ATx, out of the total number of the observed indel events of type T, n_{Tx}+n_{Mc} in the reads of both the edit and mock amplicons ATx and AMc. In this case the template statistical model a reference probability parameter q for use in the binomial distribution. The reference probability parameter q is indicative of a probability that an observed indel of type T in the reads of the edit and control collections has occurred through an edit event. As noted below this reference probability parameter q is typically set as a number close to 1 (for Edit and Mock collections of similar sizes of the same order). Then the random variable n_{Tx},n_{M}|edit is modeled with the Binomial distribution, which describes the probability of n_{Tx} successes out of n=n_{Tx}+n_{M} draws with replacement. Accordingly, without loss of generality, P(n_{Tx},n_{M}|edit) may be modeled as P(n_{Tx},n_{M}|edit)˜Binom(n_{Tx};n,q), where n=n_{Tx}+n_{M}, to obtain:

$$P ( {n}_{T x} , {n}_{M} \vert edit ) = ( \begin{matrix}{n}_{Tx} + {n}_{M} \\ {n}_{Tx}\end{matrix} ) {{q}^{{n}_{Tx}} ( 1 - q )}^{{n}_{M}} \qquad ( 3 )$$

It should be noted that the choice of reference/model parameter q to be close to unity (e.g. q for example may be chosen to be within the range of [0.92 to 0.98] or a larger range for similar number of reads of the edit and control amplicons ATx and AMc—for instance q=0.95) is based on the assumption that most of the observed indels of type T in the reads of the edit amplicons ATx amplicons are caused by an edit event of the NA edit procedure, and only a small portion in the reads of the edit and control amplicons ATx and AMc is due to background noise. Practically, the parameter q may be inferred from the experimental data and provided as reference for the system and/or method of the present invention. In this regard, it is noted that q may be a configuration parameters where higher number will increase the chances of getting false positive indication that an indels of certain types occurred due to edit event, and lower number with increase the chances of false negative indication that indels of certain types are observed due to background noise.

v. Quantifying Indel Editing Activity of the NA Editing Procedure and Optionally Determine Confidence Interval for the Same

Optionally the indel editing activity is quantified as the frequency of the edited reads out of the total number of reads in Tx:

$$\hat{p} = \frac{{n}_{T x}}{{N}_{T x}}$$

Note that this is a conservative approach as we count all reads in types classified as edits to actually represent edit events.

A confidence interval may optionally also be calculated based on the above quantification, for each potential target site using the statistical approach:

$$CI = \hat{p} \pm {Φ}^{- 1} ( 1 - \frac{α}{2} ) * \sqrt{\frac{\hat{p} ( 1 - \hat{p} )}{{N}_{T x}}} \qquad ( 4 )$$

Where \hat{p} denote the inferred editing frequency and N_{Tx} denote the total number of reads in Tx. a is the desired confidence level (which is 0.05 in our demonstration data) and c is the CDF of the standard normal distribution.

II. Statistical Model for Determining TRANSLOCATION Activity of the NA Edit Procedure

Reference is made to FIG. 1E in which a flowchart of a method 100.3 to statistically determine actual TRANSLOCATION activity of an NA edit Procedure, for one or more indel types/species, according to an embodiment of the present invention, is schematically presented. The method may be performed by the processors 1100 of the system 1000 described above. As will be appreciated by those versed in the art the operations i to iv. of the method 100.3 may be complemented by any one or more of the operations of the methods 100, 100.1 and/or 100.2 described above.

As noted above, an important advantage of the technique of the present invention is that it facilitates the detection of translocation events with fusions at on-target and off-target sites.

Since the multiplexer amplification, such as multiplex-PCR reaction, contains all primer pairs for the sites {λₘ} of interest, it is possible that amplicons will be formed based on fusion NA molecules, as the primers on both sides will be present. Accordingly, utilizing/providing reference data REF indicative of the reference NA sequences corresponding to the sites/loci of interest, λₘ₁ and λₘ the translocation activity of the NA edit procedure between these sites can be determined.

For example, determining/assessing a particular type T or species S of translocation activity of the NA edit procedure between two loci λₘ₁ and λₘ₂ where m1≠m2, according to the method 100.3 may be performed as follows:

i. Identifying Reads that are Putatively Originating from Translocation λₘ₁-λₘ₂.

This includes processing of the sequencing data ESD and MSD to determine a match between the reads thereof and the pair of sites/loci of interest, This is achieved by obtaining an ‘affected’ count matching condition of the translocations' template model. More specifically, for each read rᵢ of a plurality of the reads rᵢ^{Tx}∈R_{Tx} rⱼ^{Mc}∈R_{Mc} of the respective amplicons ATx and AMc, determine whether said read rᵢ satisfies the ‘affected’ count matching condition. The ‘affected’ count matching condition is indicative of whether a read is at least partially matching to both the reference NA sequence λₘ₁ or λₘ₂, of the pair of reference NA sequences associated with the pair of sites/loci of interest λₘ₁ and λₘ₂. The ‘affected’ count matching condition of the template model may include a combination of one or more of the following four possible translocation matching conditions DS1 to DS4, whereby each of those translocation matching conditions DS1 to DS4, is associated with a different one of four possible translocation species S:
DS1: Pfx(r) [symbol] F(λₘ₁)∧Sfx(r) [symbol] Rev(R(λₘ₂)) or a reverse-complement thereof Pfx(r) [symbol] R(λₘ₂)∧Sfx(r) [symbol] Rev(F(λₘ₁));
DS2: Pfx(r) [symbol] F(λₘ₂)∧Sfx(r) [symbol] Rev(R(λₘ₁)) or a reverse-complement thereof Pfx(r) [symbol] R(λₘ₁)∧Sfx(r) [symbol] Rev(F(λₘ₂));
DS3: Pfx(r) [symbol] F(λₘ₂)∧Sfx(r) [symbol] Rev(F(λₘ₁)) or a reverse-complement thereof Pfx(r) [symbol] F(λₘ₁)∧Sfx(r) [symbol] Rev(F(λₘ₂));
DS4: Pfx(r) [symbol] R(λₘ₂)∧Sfx(r) [symbol] Rev(R(λₘ₁)) or a reverse-complement thereof Pfx(r) [symbol] R(λₘ₁)∧Sfx(r) [symbol] Rev(R(λₘ₂));
   whereby F(λₘ) and R(λₘ) respectively designate the prefix and suffix primer sequences PRS⁺ₘ and PRS⁻ₘ of the respective site λₘ, and Pfx(r) and Sfx(r) respectively designate prefix and suffix of a read r, [symbol] denotes a best match (e.g. according to lowest edit distance between the prefix/suffix of the read and prefix/suffix primer sequences of the plurality of sites {λₘ}₁^{M}. and the reverse complements of said primer sequences), and Rev(denotes a reverse-complement function of a nucleic acid sequence.

FIG. 1F is a schematic self-explanatory illustration of all four possible translocation species depicted for example sites λₘ₁ and λₘ₂ of lengths of about 300 base pairs in the respective p arms of chromosomes 3 and 10, CH3 and CH10; The figure schematically illustrates the four possible translocations species A, B, C and D between these sites λₘ₁ and λₘ₂ of chromosomes 3 and 10 p arms, CH3 and CH10. The expected cut-positions i₀ of NA editing at these sites are marked by lines of red color in the respective chromosomes. The four possible translocations species A, B, C and D are depicted where ⊕ denotes the concatenation/fusion between the left and/or right parts of the NA sequences of the respective sites λₘ₁ and λₘ₂. The four possible fusion types give rise to structures that are either single-centromeric (A & B), centromere-free (C), or double-centromeric (D).

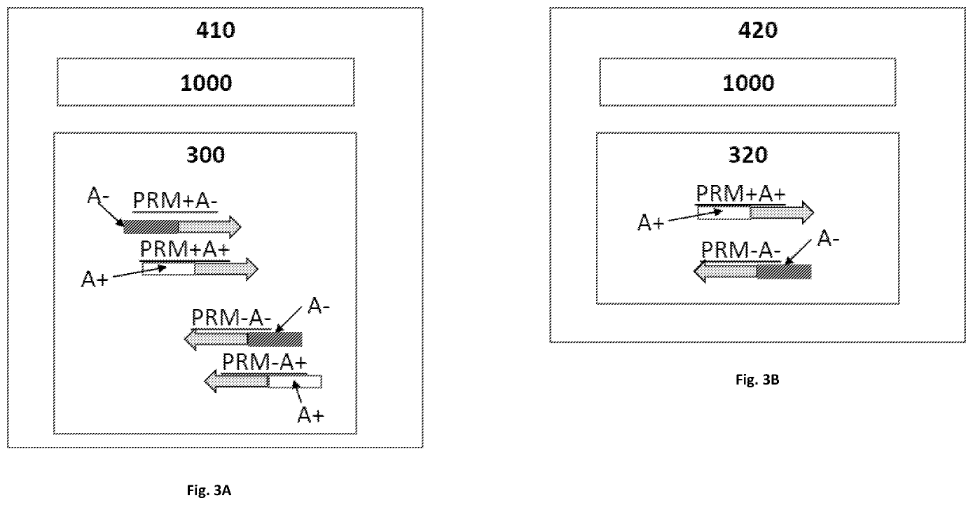

More specifically, the translocations species A, B are single-centromeric formed respectively by fusion of (A) the left-part L of the site λₘ₂ in chromosome CH10 and right-part R of the site λₘ₁ in chromosome CH3 (i.e. 10-L⊕3-R), and vice versa (B) the left-part of the site λₘ₁ in chromosome CH3 and right-part of the site λₘ₂ in chromosome CH10 i.e. 3-L⊕10-R). The single-centromeric translocations species A, B can be identified by the respective translocation matching conditions DS1 and DS2 indicated above. More specifically, the translocations species C, D are centromere-free (C) and double-centromeric (D) formed respectively by fusion of (C) the left-part L of the site λₘ₂ in chromosome CH10 and left-part L of the site λₘ₁ in chromosome CH3 (i.e. 10-L⊕3-L), and (D) the right-part of the site λₘ₁ in chromosome CH3 and right-part of the site λₘ₂ in chromosome CH10 (i.e. 3-R⊕10-R). The centromere-free C and the double-centromeric D translocations species may be identified by the respective translocation matching conditions DS3 and DS4 indicated above (provided that a pairs of primers with suitable sequencing adapters, as described in the present invention are used in the amplification, see e.g. FIGS. 3B and 3C below).

To this end the ‘affected’ count matching condition of the template model may include any one DSj of the above translocation matching conditions DS1 to DS4, in case a selected species j of translocation is to be separately identified/assessed; or it may be composed as a combined condition combined from two or more of the above translocation matching conditions DS1 to DS4 in alternative form. In the latter case for example in case all species of translocations are to be assessed/determined without distinction, the combined condition DS will be satisfied by a read in case any of the translocation matching conditions DS1 or DS2 or DS3 or DS4 is satisfied.

The ‘affected’ count matching condition is used to identify dual site partially matching collections C_{TX}(λₘ₁,λₘ₂), C_{Mc}(λₘ₁,λₘ₂) of reads of the amplicons ATc and AMc of the edit and mock collections respectively. These reads, which are found to match one or more of the translocation matching conditions for the pair of sites (λₘ₁,λₘ₂) are asserted as reads representing the putative amplicon that represents the λ₁ to λ₂ fusion at the cut site.

Optionally the dual site partially matching collections C_{TX}(λₘ₁,λₘ₂), C_{Mc}(λₘ₁,λₘ₂) of reads are further filtered by performing an alignment of the reads to a putative reference amplicon sequence that represents the λ₁ to λ₂ fusion at their respective cut sites (as determined by the PAM) and retaining only those reads which have sufficiently strong alignment scores, e.g. above a certain alignment threshold.

The reads of the collections C_{TX}(λₘ₁,λₘ₂), C_{Mc}(λₘ₁,λₘ₂) which are found to match one or more of the translocation matching conditions for the pair sites (λₘ₁,λₘ₂) and which are possibly also aligned with sufficiently high alignment score to the putative reference amplicon sequence representing the λ₁-λ₂ translocation, are considered as reads attesting to fusion of the pair of sites (λₘ₁,λₘ₂).

Accordingly the counts n_{Tx} and n_{Mc} of the number of reads in these respective collections C_{TX}(λₘ₁,λₘ₂), C_{Mc}(λₘ₁,λₘ₂) that represent the respective ‘affected’ counts n_{Tx} and n_{Mc} of reads, in which a translocation involving fusion of both site [λₘ₁,λₘ₂], are observed. In other words, for translocations, the ‘Affected’ counts n_{Tx} and n_{Mc} are determined as the respective sizes of the dual site partially matching collections such that n_{Tx}=|C_{TX}(λₘ₁,λₘ₂)| and n_{Mc}=|C_{Mc}(λₘ₁,λₘ₂)|.

ii. Identifying Reads that Represent Amplicons Originating from Either of the Sites λₘ₁, λₘ₂.

This includes processing the sequencing data ESD and MSD to determine the ‘collective’ read counts C_{TX}(λₘ₁) and CM(λₘ₂). C_{TX}(λₘ₁) counts the reads that have either end matching a primer from the primer pair of [λₘ₁,λₘ₂]. To this end optionally the single site partially matching condition with a site λₘ will be considered as satisfied for each read that either end thereof (prefix or suffix) matches the prefix or suffix sequence of the respective site. C_{TX}(λₘ₂) counts the reads that have either end matching a primer from the primer pair of λₘ₂. We thereby get the four counts C_{TX}(λₘ₁), C_{TX}(λₘ₂), C_{Mc}(λₘ₁), C_{Mc}(λₘ₂) of reads of the edit and mock amplicons ATx and AMc.

The ‘collective’ counts can be separately counted for the four different translocation species. These are preferably obtained from the above numbers, C_{TX}(λₘ₁), C_{TX}(λₘ₂), C_{Mc}(λₘ₁), C_{Mc}(λₘ₂), by dividing them by the total number or possible species, 4, and possibly multiplying by the number of species actually considered.

The sizes of the single site partially matching collections {C_{TX}(λₘ₁), C_{TX}(λₘ₂)}, {C_{Mc}(λₘ₁),C_{Mc}(λₘ₂)} provided the total number of relevant reads, in both Tx and Mc collections, and are indicative of the respective ‘collective’ counts N_{Tx} and N_{Mc} of reads of amplicons.

For example, the ‘collective’ counts N_{Tx} and N_{Mc} are estimated based on respective sizes of the following pairs of single site partially matching collections [|C_{TX}(λₘ₁)|, |C_{TX}(λₘ₂)|], [|C_{Mc}(λₘ₁)|, |C_{Mc}(λₘ₂)|]. For example the ‘collective’ counts N_{TX} and N_{Mc} of for translocations may be estimated as respective averages of the respective sizes of the pairs of single site partially matching collections of each of the edited and mock collections as follows: N_{Tx}=<|C_{TX}(λₘ₁)|, |C_{TX}(λₘ₂)|> and N_{Mc}=<|C_{Mc}(λₘ₁)|, |C_{Mc}(λₘ₂)|> (where < > indicates average). In a particular example the ‘collective’ counts N_{Tx} and N_{Mc} are estimated as respective geometrical averages of the respective sizes of the pairs of single site partially matching collections.

iii. Applying Aa Statistical Classification Model to Determine Whether Reads Associated with the Translocation Type T Originate from an Actual Translocation Editing Activity of Translocation Type T or Species S

As indicated above, the technique of the present invention facilitates distinguishing reads resulting from edit events of the NA edit procedure from translocation reads derived from various sources of noise (e.g. from the amplification or sequencing). This distinction is based on the ability to assess whether a specific translocation type/species originates from edit event of the NA edit procedure or is a result of experimental background noise (such as sequencing artifacts or erroneous read assignments). The model used according to an embodiment of the present invention for this distinction, is based on Probability Distribution suitable for the statistics of translocation in the amplicons ATx of the edited collection vs the amplicons of the control/mock collection AMc of the edited collection. A person of ordinary skill in the art would readily appreciate after knowing the present invention, that other statistical models may also be suitable for the purpose of the present invention.

The template statistical model provided for assessing the TRANSLOCATION activity of the NA editing procedure includes a statistical classifier adapted to classify whether translocation events of certain types T or species S originate from NA editing.

For each one or more of translocation classes/types T=[λₘ₁, λₘ₂], and possibly for each species S of one or more species thereof, the system 1000 or method 100 according to an embodiment of the present invention, applies the classifier to classify whether translocation events of this type T and possibly species S, are a result of an editing event or are noise (such as amplification/PCR noise or sequencing noise which also occur in the mock sample).

The above ‘affected’ counts and ‘collective’ counts are computed for each such type T or species S of translocation which is to be determined. Using these counts the classifier is applied/computed to determine whether the translocation of this type/species occur due to edit events of the NA editing procedure (e.g. determine the probability of such occurrence).

In some embodiments the classifier used for translocations is a Probability Distribution function.

In a particular none limiting example, a hypergeometric tail distribution function HGT is used as the classifier for translocations.

$$H G T ( b ; N , B , n ) = {\sum }_{i = b}^{min ( n , B )} \frac{( \begin{matrix}B \\ i\end{matrix} ) ( \begin{matrix}N - B \\ n - i\end{matrix} )}{( \begin{matrix}N \\ n\end{matrix} )} \qquad ( 5 )$$

The inventors of the present invention have found that hypergeometric tail may be used to determine with good accuracy, whether a translocation of this type T or species S is likely to have occurred due to the NA editing procedure, and more specifically optionally to statistically assess/determine whether this type T or species S, of translocation has occurred due to the NA editing procedure. The assessment of the TRANSLOCATION activity of at least one type T or species S may be determined by computing the probability of the hyper geometric tail distribution based on the respective combined ‘collective’ counts N_{Tx} and N_{Mc}, and combined ‘affected’ counts n_{Tx} and n_{Mc} of reads of amplicons which are associated with the respective translocation type T or species S. This may be performed as follows for each particular type/class, and possibly species of S of translocation of interest:

Consider a possible translocation type T between target sites λₘ₁ and λₘ₂. The parameters of the hypergeometric tail distribution function HGT may be set based on the above indicated collective’ counts N_{Tx} and N_{Mc}, and ‘Affected counts n_{Tx} and n_{Mc} determined for the particular type T or species S of the translocation, as follows:
b and B are respectively the ‘Affected’ counts of the edit collection and the total Affected counts of this type T or species of translocation:

b=n_{Tx}=C_{TX}(λm1,λm2);

B=n_{Tx}+n_{Mc}=CTX(λm1,λm2)+CMc(λm1,λm2)
n and N may be computed based on the collective counts N_{Tx} and N_{Mc} of this type T or species S of translocation (which are obtained as averages of the numbers {C_{TX}(?λₘ₁), C_{TX}(λₘ₂)} and {C_{Mc}(λₘ₁),C_{Mc}(λₘ₂)}), as follows:

n=N_{Tx}≅\sqrt{|C_{Tx}(λₘ₁)|·|C_{Tx}(λₘ₂)|}

N=N_{Tx}+N_{Mc}≅\sqrt{|C_{Tx}(λₘ₁)|·|C_{Tx}(λₘ₂)|}+\sqrt{|C_{Mc}(λₘ₁)|·|C_{Mc}(λₘ₂)|}

Accordingly, the probability P-value of the translocation of this type T or species S of translocation occurring due to an edit even, may be determined based on the hyper geometric tail function in equation (5 above) based on these parameters.

iv. Quantifying the Actual TRANSLOCATION Activity of the NA Editing Procedure and Optionally Determine Confidence Interval for the Same:

As indicated above, according to various embodiments of the present invention the translocation classification in operations (i) to (iii) above may be carried out for one, several or all observed types/classes T and possibly species S of translocations observed in the reads. Accordingly, a list of p-values may be obtained for all considered translocation types/species.

Thus optimally, a rate and a confidence interval of the translocation activity of the NA editing procedure may be determined/quantified for a given translocation type T or, possibly, species S. by computing

$$\hat{p} = \frac{C T X ( λ m 1 , λ m 2 )}{{N}_{T x}}$$

Where N_{Tx}=\sqrt{|C_{Tx}(λₘ₁)|·|C_{Tx}(λₘ₂)|} is the collective read counts average for the two pertinent sites.

For a single species translocation S the number

N_{Tx}=¼\sqrt{|C_{Tx}(λₘ₁)|·|C_{Tx}(λₘ₂)|}

may be used.

And

$$CI = \hat{p} \pm {Φ}^{- 1} ( 1 - \frac{α}{2} ) * \sqrt{\frac{\hat{p} ( 1 - \hat{p} )}{{N}_{T x}}}$$

Optionally, the list of p-values may then be FDR³⁸ corrected (i.e. corrected for False Discovery Rate as would be appreciated by those versed in the art) to filter out translocations with FDR above a certain predetermined FDR threshold (e.g. FDR-threshold=0.05).

The list of p-values (e.g. those with FDR above the FDR threshold) may then be output as a lists of translocation types and possibly species thereof and the respective probabilities (P-values) of their occurrence of due to the NA edit procedure.

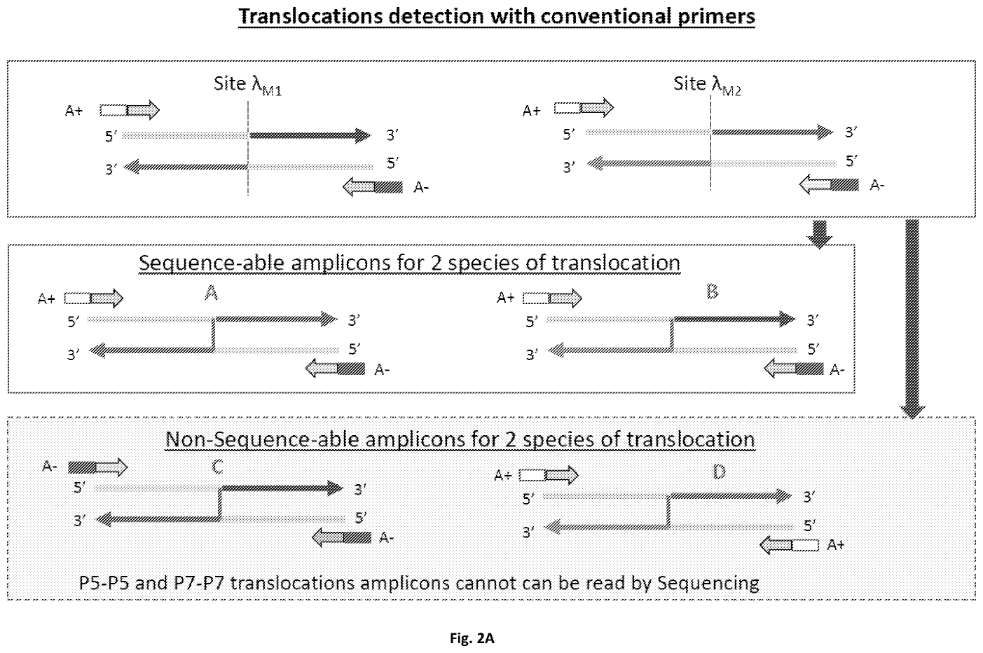

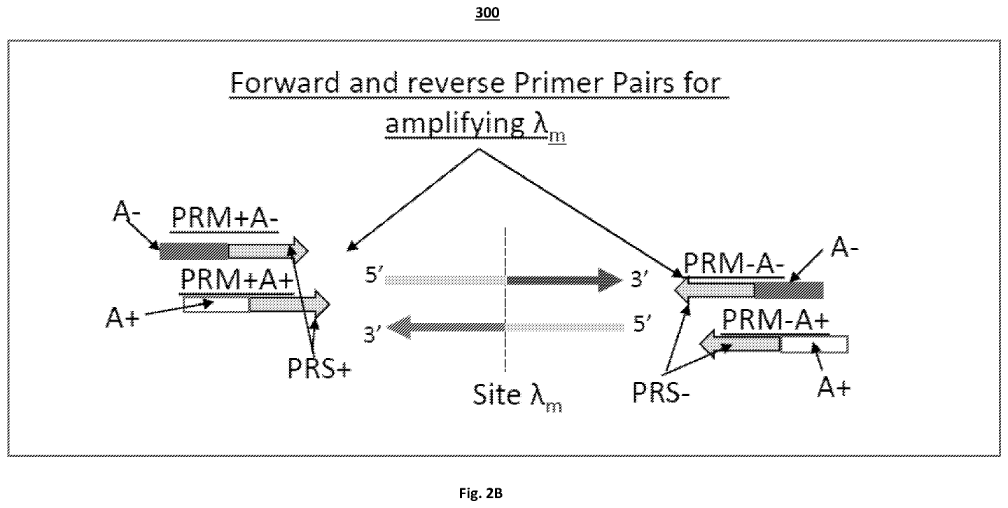

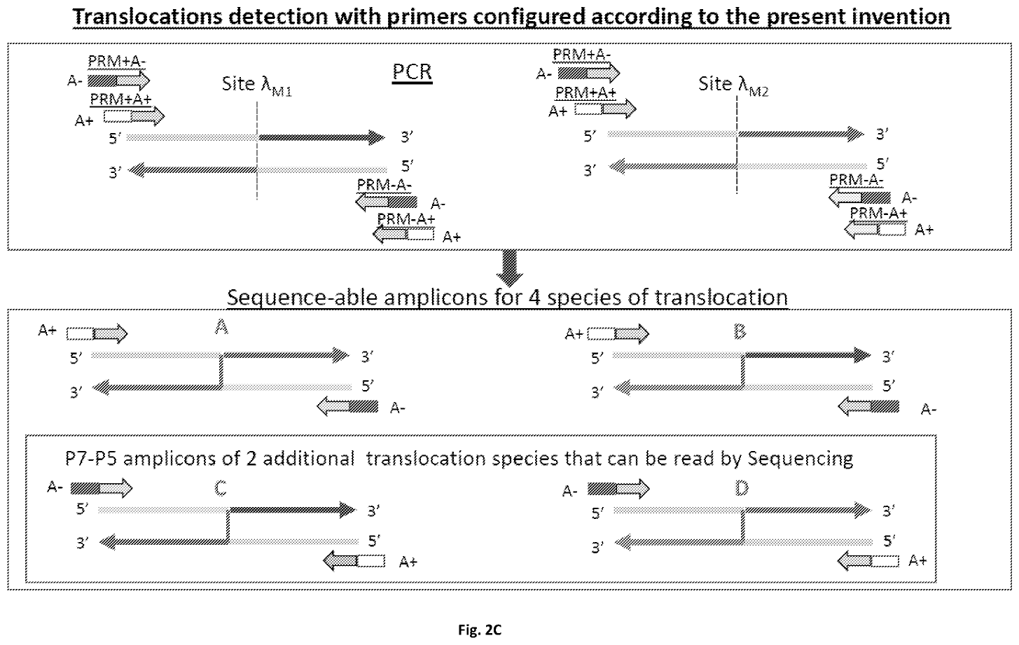

Reference is now made together to FIGS. 2A to 2C in which: FIG. 2A illustrates the conventional PCR amplification techniques which are not adapted for producing amplicons suitable for sequencing for certain translocation species; FIG. 2B schematically illustrates a kit comprising a set of a plurality of primer molecule types {PRₜ} four use in multiplex amplification to yield amplicons suitable for sequencing for all four translocation species to thereby enable determining the translocation effects of a NA editing procedure; FIG. 2C schematically illustrates a multiplex amplification (which may be carried out in either a one-step or two-step multiplex amplification) with the primer kit according to an embodiment of the present invention and the translocation amplicon products thereof which facilitate the sequencing of all four translocation species.

To this end, FIG. 2A illustrates a conventional PCR amplification with a conventional primer kit. The conventional kit includes a set of a plurality of primer molecule types {PRₜ} designed to provide amplification of one or more sites {λₘ}. In the conventional kit the plurality of primer molecule types {PRₜ} include one or more matched pairs (PRM⁺A⁺, PRM⁻A⁻) of forward PRM⁺ and reverse PRM⁻ primer molecule types suitable for amplification of the one or more sites {λₘ}.

In the conventional techniques each respective forward primer PRM⁺A⁺, PRM⁺A⁺) includes a forward binding primer sequence and a forward adapter sequence A+, and each respective reverse primer includes a revers binding primer sequence and a reverse adapter sequence A−.

In this regard it should be appreciated that the term adapter is used herein to indicate either amplification adapter (as generally in a 2-step multiplex amplification process Two-Step-PCR including steps PCR1 and PCR2 briefly described below) or a sequencing adapter (as generally used in either the 1-step multiplex amplification process, One-Step-PCR, or in the second step PCR2 of the 2-step multiplex amplification process, in order to enable sequencing of the amplification products.

Conventional One-Step-PCR/Amplification

Briefly as generally known, the primer molecule types {PRₜ} used in a 1-step multiplex amplification process, include matched pairs (PRM⁺ₘ, PRM⁻ₘ) of forward PRM⁺ₘ and reverse PRM⁻ₘ primer molecule types per each site λₘ of the one or more sites {λₘ} that are to be amplified, the forward binding primer sequence PRS+ₘ of the forward primer PRM⁺ₘ of the site m includes a an NA sequence complementary to the site's λₘ prefix sequence and the revers binding primer sequence PRS−ₘ of the revers primer PRM⁻ₘ of the site m includes a NA sequence complementary to the site's λₘ suffix. The respective forwards and reverse adapters, A+ and A−, in this case are typically respectively forwards and reverse sequencing adapters, such as the generally known P5 and P7 adapters.

Conventional Two-Step-PCR/Amplification

In a Two-Step-PCR the primer molecule types {PRₜ} used in the 1^{st} step PCR1, include, as in the One-Step-PCR, matched pairs (PRM⁺ₘ, PRM⁻ₘ) of forward PRM⁺ₘ and reverse PRM⁻ₘ primer molecule types per each site λₘ of the one or more sites {λₘ} that are to be amplified, the forward binding primer sequence PRS+ₘ of the forward primer PRM⁺ₘ of the site m in this case includes an NA sequence complementary to the site's λₘ prefix sequence and the revers binding primer sequence PRS−ₘ of the revers primer PRM⁻ₘ of the site m includes an NA sequence complementary to the site's λₘ suffix. However here, the respective forwards and reverse adapters, A+ and A−, are forwards and reverse amplification adapters, which are needed-for/used-in the 2^{nd} step PCR2, and serve as site prefix and suffix for binding the primers of the 2^{nd} step PCR2 (e.g. the forwards and reverse amplification adapters used in the 1^{st} step may be universal for all, or a plurality, of the sites {λₘ}, so that the forwards and reverse primer molecule types used in the 2^{nd} step PCR2 may be insensitive/non-specific to the particular sequences of the sites {λₘ}). Accordingly, the 2^{nd} step PCR2 may be conducted with as little as a single type of matched pair (PRM⁺, PRM⁻) of forward PRM⁺ and reverse PRM⁻ primer molecules (e.g. a universal matched pair). The forward primer molecule PRM⁺ of the 2^{nd} step PCR2 includes a forwards sequencing adapter (e.g. P5) and a forward binding primer sequence PRS+ complementary for binding to the forward amplification adapter used in the 1^{st} step (e.g. non-site-specific), and accordingly the reverse primer molecule PRM⁻ of the 2^{nd} step includes a revers sequencing adapter (e.g. P7) and a revers binding primer sequence PRS− complementary for binding to the reverse amplification adapter used in the 1^{st} step (e.g. also non-site-specific).

In view of the above amplification's products of each site of interests λₘ, which are produced by either the above described conventional One-Step-PCR or Two-Step-PCR multiplex PCR amplifications, include amplicons of the site of interest with two match pairs of sequencing adapters (e.g. P5 and P7), from either side of the site's λₘ amplicon. This facilitates sequencing of the site's amplicons since such configuration of the sequencing adapters from either side thereof are required for the sequencing process, particularly for NGS sequencing.

However, in implementations of the Conventional multiplex PCR process (e.g. the One-Step-PCR or Two-Step-PCR processes described above) for detection of translocations, not all the translocation species will be produced/amplified with the amplicons having the suitable arrangement of forwards and revers sequencing adapters from either side thereof. Indeed, as illustrated in the figure only the single-centromeric translocations species A and B, which are described above will be amplified with the required forwards A+ and revers A− sequencing adapters from either side thereof, while the centromere-free translocations species C and double-centromeric translocations species D described above will be amplified with either the forward or the revers sequencing adapters appearing from both sides thereof. Accordingly, the centromere-free translocations species C and the double-centromeric translocations species D will not be sequenced with the conventional simplification techniques.

To overcome this deficiency of the conventional techniques, the present invention, in some embodiments thereof, provides a kit 300 for determining effects of a NA editing procedure, the kit 300 includes a set of a plurality of primer molecule types {PRₜ} designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites.

In this regard it should be understood that in the scope of FIGS. 2A to 2C the phrase primer molecule types designed/suitable for amplification of the one or more sites {λₘ} pertains to respective forward and reverse primers having respective primer forward and reverse primer binding sequences PRS+ and PRS−, that are suitable for binding to NA molecules, which include the sequences of respective sites {λₘ}, to allow their amplification. To this end the forward and reverse primer binding sequences, PRS+ and PRS−, may be complementary to the respective sites prefix and suffix sequences of the respective sites {λₘ}, (e.g. in case the primers are used of a One-Step multiplex amplification of for the 1^{st} step of a Two-Step multiplex amplification), or the forward and reverse primer binding sequences PRS+ and PRS− may be complementary to respective forward and reverse amplification adapters (not specifically shown) used in a in case the primers are used for the 1^{st} step of a Two-Step multiplex amplification.

To this end, the set/kit 300 of the plurality of primer molecule types {PRₜ} according to an embodiment of the present invention includes pairs (PRM⁺, PRM⁻) of forward PRM⁺ and reverse PRM⁻ primer molecule types {(PRM⁺, PRM⁻)∈{PRₜ} suitable for amplification of said on-target and off-target sites {λₘ}₁^{M} such that each respective forward and revers primer molecule, PRM⁺ and PRM⁻, include at least one of a forward and revers adapters, A+ and A− (e.g. P5 and P7 in case of sequencing adapters or other types of adapters of example forward and revers amplification adapters);

The set/kit 300 of the plurality of primer molecule types {PRₜ} is characterized in that the plurality of primer types {PRₜ} includes:
forward primer molecules PRM^{+A+} including forward adapters A+;
forward primer molecules PRM^{+A-} including revers adapters A−;
reverse primer molecules PRM^{-A+} including forward adapters A+;
reverse primer molecules PRM^{-A-} including revers adapters A−;

thereby enabling sequencing of all possible translocation species between at least one pair of the editing sites {λₘ}₁^{M}.

The Kit 300 may be configured and operable for use in any suitable Multiplex-Amplification process.

To this end, in some embodiments the kit may be configured for use in One-Step-Multiplex Amplification process or a 1^{st} step, PCR1, of a Two-Step-Multiplex-Amplification process. The primer molecule types {PRₜ} of the Kit 300 in this case, include pairs (PRM⁺ₘ, PRM⁻ₘ) of forward PRM⁺ₘ and reverse PRM⁻ₘ primer molecule types, per each site λₘ of the one or more sites {λₘ} that are to be amplified. In this case the forward binding primer sequence PRS+ₘ of the forward primer PRM⁺ₘ of the site m includes an NA sequence complementary to the site's λₘ prefix sequence and the revers binding primer sequence PRS-m of the revers primer PRM⁻ₘ of the site m includes an NA sequence complementary to the site's λₘ suffix.

Specifically in such embodiments, the Kit 300 may be configured for use in a 1^{st} step, PCR1, of a Two-Step-Multiplex-Amplification process. The forward and reverse adapters, A+ and A−, in this case are forwards and reverse amplification adapters facilitating that all said translocation species will be amplified in said 2^{nd} step, PCR2, of the Two-Step-Multiplex-Amplification process, to produce amplicons thereof which have forwards and revers sequencing adapters from either side of the amplicon.

Alternatively, or additionally, the Kit 300 may be configured for use in One-Step-Multiplex Amplification process. In this case, the forwards and reverse adapters A+ and A− are forwards and reverse sequencing adapters.

In some embodiments the Kit 300 may be configured for use in a 2^{nd} step, PCR2, of a Two-Step-Multiplex-Amplification process. In such embodiments the primer molecule types {PRₜ} include pairs (PRM⁺, PRM⁻) of forward PRM⁺ and reverse PRM⁻ primer molecule types including respective forward PRS+ and reverse PRS− binding primer sequences complementary to respective forwards and reverse amplification adapters (not shown) of a 1^{st} step, PCR1, of the Two-Step-Multiplex-Amplification process. In this case the forwards and reverse adapters, A+ and A−, of the forward PRM⁺ and reverse PRM⁻ primer molecule types, are forwards and reverse sequencing adapters.

FIG. 2C schematically illustrates a multiplex PCR/amplification carried out with the primer kit 300 according to the present invention as described above. The amplification products of each translocation species between pairs of site of interests [λₘ, λₘ₂] which are produced the multiplex amplifications with the kit of the present invention, include all four species pf translocation amplicons of the sites of interest (as far as such translocations exist), with two match pairs of forwards and revers adapters A+ and A− (e.g. sequencing adapted such as P5 and P7 or amplification adapters), from either side of the translocation amplicons of each of the different 4 species: A, B, C and D. This facilitates sequencing of all 4 translocation species to observe/determine whether an NA edit procedure causes any of these species.

Reference is now made to FIGS. 3A and 3B illustrating in a block diagram two embodiments of kits 410 and 420 for determining adverse effects of an NA editing procedure according to two embodiments of the present invention. The kit 410 illustrated in FIG. 3A includes the system 1000 of the present invention according to an embodiment of the invention, and a conventional primer kit/set 320 including pairs of forward and reverse primers PRM⁺A⁺ and PRM−A− with respective forward and reveres adapters A+ and A−. The kit 420 illustrated in FIG. 3B includes the system 1000 of the present invention according to an embodiment of the invention, and a primer kit/set 300 according to the present invention as described above.

As indicated above, the system 1000 may be include a non-transitory computer readable medium storing instructions executable by a processor, for utilizing the sequencing products of any of these kits to determine and output data indicative of the effects of Nucleic Acid (NA) editing procedure according to any of the methods described above according to the present invention.

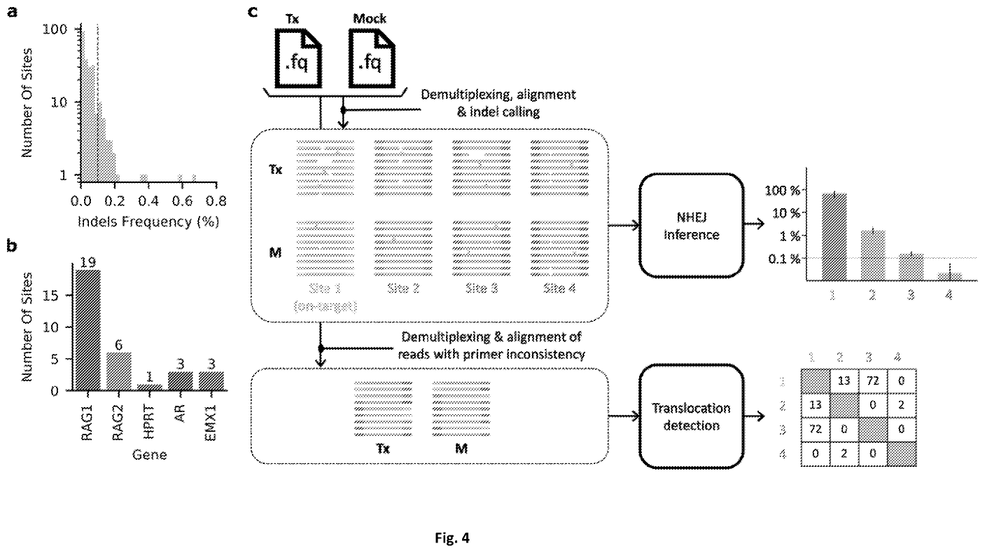

Several tests were performed to assess the accuracy of the technology of the present invention in determining the occurrence of various adverse effects of NA editing procedures. FIG. 4 is a self-explanatory illustration depicting some test for detecting quantifying off-target activity according to some embodiments of the present invention. Sections (a) and (b) of this figure graphically illustrate hypothetical indels at the expected cut-site in the Mock sequencing data. The technique of the present invention was applied to multiplex-PCR NGS data produced from five different gRNAs, corresponding to five on-target genomic loci, and covering 226 off-target sites, under different conditions. With reference to the graph of Section (a), out of 226 off-target sites that were examined 31 sites were found to have indel frequency higher than 0.1% (fraction=0.001) in the Mock sequencing data. The indel frequencies in the Mock sequencing data, at the cut-site were measured, by a direct calculation, not using a comparative set-up. With reference to the graph of Section (b), this graph presents 31 noisy off-target sites that come from different gRNAs examined, as depicted. Section (c) schematically illustrates an example workflow of the system 1000 according to an embodiment of the present invention. The system 1000 assigns each read in the Tx and Mock FQASTQ sequencing data (indicated .fq files), to a specific locus of interest or a putative translocation. Then, a Bayesian inference classifier was applied to accurately estimate the indel editing activity, and a hypergeometric test is performed to detect translocation reads.

Some experiments use a generally known rhAmpSeq assay (IDT, Coralville, IA) for the PCR and were conducted with a Tx vs M design. These yielded a total of 1,161 instances. The results obtained from evaluating the indel and translocation activity by applying the technique of the present invention to the sequencing data from these experiments, shown that the technique of the present invention accurately estimates indel activity levels at off-target sites.

Indeed, the editing activity estimation according to the present invention is based on the ability to model the background noise while being blind/insensitive to the source of the noise, whether it comes from high NGS error rates (FIG. 1F is a schematic self-explanatory illustration of all four possible translocation species depicted for example for chromosomes 3 and 10 p arms;

FIG. 2A illustrates conventional PCR amplification techniques which are not adapted for producing amplicons suitable for sequencing for certain translocation species

FIGS. 2B and 2C schematically illustrates a multiplex amplification according to embodiments of the present invention capable of producing amplicons suitable for sequencing of all four translocation species; in which FIG. 2B illustrates a primer kit according to the present invention for use in multiplex amplification to yield the four translocation species; and FIG. 2C schematically illustrates a multiplex amplification (which may be carried out in either a one-step or two-step multiplex amplification) with a primer kit according to the present invention for producing translocation amplicons that facilitate the sequencing of four translocation species;

FIGS. 3A and 3B are block diagrams illustrating two respective kits 410 and 420 for determining adverse effects of an NA editing procedure according to two embodiments of the present invention;

(a-b)), false site assignments, or ambiguous alignments. The template classifier applied to the results of each experiment statistically models the background noise thereby quantify editing events.

The performance of technique of the present invention were tested on the challenging off-target scenarios, where high error rates occur at sites with low editing activity, as well as in scenarios where process related (NGS, PCR, etc.) error rates can lead to false-negative inferences. The results of these test showed the technique of the present invention can recover the true validated editing activity even when error and editing rates are near identical, where the true validated editing activity was obtained by human examination of the actual reads as well as by two lines of statistical evidence as explained above.

Accurate editing activity estimation may also depend on the detection of alternative cut-sites. Flawed identification of the off-target gRNA binding configuration, due to an ambiguous alignment or false interpretation of GUIDE-seq or other screening methods, can lead to a mis-inferred PAM position. Moreover, optimal read alignments, even if slightly better justified from a biochemical perspective, can place real edit events away from the expected cut-site. Finally, real editing activity can occur away from the expected cut-site due to the existence of an alternative PAM sequence or less frequent non-canonical DSB mechanisms. The technique of the present invention facilitates the detection of alternative cut-sites by incorporating different prior probabilities for each position in the reference sequence, as described above. As indicated above, one novel and inventive feature of the technique of the present invention lies in is its ability to detect translocations resulting from NA/CRISPR editing procedures, by analyzing NGS data produced by a multiplex PCR using locus-specific primers (for instance such as rhAmpSeq³⁴). Using the multiplex PCR mechanism for target enrichment (with primers designed to span the potential cut-positions of the off-target sites), four species of translocation events can occur for every pair of potential partner loci. The technique of the present invention is capable of analyzing the mixed pairs of primer sequences that are detected on common reads in the NGS data. These reads represent putative fusion amplicons which may be due to translocations by NA edit procedure. A statistical model (e.g. hypergeometric) may be applied according to the present invention, to those reads to infer there statistical significance and determine those who most probably pertain to translocations with significant (FDR corrected) p-values (≤0.05, by default).

In several tests conducted with the technique of the present invention, significant translocations were detected. For example, for editing procedures conducted on RAG1 and RAG2 loci in HEK293-Cas9 cells¹², the technique of the present invention revealed evidence for translocations in 20 and 19 unique pairs of sites, for RAG1 and RAG2, respectively. The most significant corrected p-values are 4.9*10⁻²² for on-target site 1 with off-target site 7 in RAG1, and 1.53*10⁻⁵³ for off-target site 1 with off-target site 5 in RAG2.

These results were experimentally confirmed in an independent measurement using the singleplex droplet digital PCRS using primers designed to separately amplify individual potential translocation events.

It is noted that occurrences of all four possible configuration species of translocations for different pairs of loci were detected including centromere-free, double-centromeric, and two single-centromeric configurations. This was achieved by using a multiplex amplification with the primer kit 300 as described above, and particularly using PCR panel with both P5 and P7 on both the reverse and forward primers, to enable measuring all four types of translocations at every potential fusion site covered by the assay. In conclusion, the technique of the present invention provides significant improvement in the accuracy of determination of genome/NA-editing adverse effects to enhance and accelerate the sound and accurate broader use of NA editing in biotechnology and therapeutic applications. A person of ordinary skill in the art will readily appreciate the various modification which can be implemented to the above describes systems and methods without departing from the scope of the present invention as defined by the claims.

1. A method for determining effects of Nucleic Acid (NA) editing procedure, the method includes:
providing a first and second collections of NA sequences originated from the same NA source, whereby the first collection is an edited collection of NA sequences from said NA source to which a certain NA editing procedure was applied, and the second collection is a control (mock) collection of NA sequences of said NA source to which said NA editing procedure was not applied;
providing target data indicative of expected editing sites {λₘ}₁^{M} of the NA editing procedure including at least one on-target site {λ₁} and one or more off-target sites {λₘ}₂^{M}, where λₘ represents an off-target or on-target site indexed m and M is a number of the expected on-target and off-target sites;
applying multiplexed amplifications to the edited and control collections respectively, and thereby obtaining respective amplified products/amplicons of said edited and control collections, whereby the multiplexed amplifications of the edited and control collections are conducted with similar primer molecule types;
sequencing the multiplexed amplifications products/amplicons of said edited and control collections to obtain sequencing data indicative of pluralities of reads, R_{Tx}={rᵢ^{Tx}} and R_{Mc}={rⱼ^{Mc}}, of said multiplexed amplifications' products/amplicons from each of the edited and control collections;
processing said sequencing data by a processor for constructing, per each particular type of adverse effect of one or more types of possible adverse effects of said NA editing procedure, a statistical model of occurrence of said type of adverse effect by the NA editing procedure, and applying said statistical model to said sequencing data to statistically determine actual occurrence of said type of adverse effect by the NA editing procedure; and
outputting data indicative of the of whether said each type of adverse effect by actually occurs due to the NA editing procedure, to thereby enable determination of safety of the NA editing procedure.

2. The method of claim 1, wherein said processing further comprising utilizing the statistical model to quantify the types of adverse effects actually affected by the NA editing procedure by determining rates of occurrence thereof by the NA editing procedure, and a statistical confidence intervals for said rates.

3. The method of claim 1, wherein said one or more types of adverse effect are classified to one or more classes of adverse effects, each class being characterized by the one or two participating sites [λₘ₁,λₘ₂], with which adverse effects of the class are associated, whereby each class belongs to one of two categories of adverse effects:
Category 1 (INDELs): adverse effects involving one site [λₘ₁₌=λₘ₂] where m1=m2; and
Category 2 (TRANSLOCATIONs): adverse effects involving two sites [λₘ₁,λₘ₂] where m1≠m2; and
wherein said processing said sequencing data for the particular type of adverse effect comprises carrying out the following operations a. to d. for at least one of said two categories of adverse effects:
a. providing a template statistical model corresponding to the category of said particular type of adverse effect;
b. processing the sequencing data according to the class [λₘ₁,λₘ₂] of said particular type of adverse effect to determine or assess respective ‘collective’ counts N_{Tx} and N_{Mc} of reads of amplicons which are associated with the participating sites [λₘ₁,λm₂] of said class in the sequencing data of each of the edited and control collections;
c. processing the sequencing data according to the particular type of adverse effect to determine or assess respective counts n_{Tx} and n_{Mc} of ‘affected’ reads of amplicons in which said particular type of adverse effect is observed;
d. applying said template statistical model to the respective ‘collective’ counts N_{Tx} and N_{Mc} of reads of amplicons which are associated with the participating sites [λₘ₁,λₘ₂] in the edited and control collections and to the respective ‘affected’ counts n_{Tx} and n_{Mc} of reads of amplicons in which said particular type of adverse effect is observed, from amongst the reads of amplicons which are associated with the edited and control collections; and
thereby obtaining the said statistical determination of the occurrence of said particular type of adverse effect by the NA editing procedure (e.g. in tested cell types or in related samples and/or in clinical material).

4. The method of claim 1, wherein said multiplexed amplifications of the edited and control collections are conducted utilizing respective multiplex PCR processes with a similar selected set of primer molecule types{PRₜ}; and wherein the method includes providing the selected set of a plurality of primer molecule types {PRₜ} including primer molecule types selected according to said target data, such that the plurality of primer types {PR} comprise, or constitutes of, matched pairs (PRM⁺ₘ, PRM⁻ₘ) of forward PRM⁺ₘ and reverse PRM⁻ₘ primer molecule types {(PRM⁺ₘ, PRM⁻ₘ)}₁^{M}∈{PRₜ} suitable for amplification of said on-target and off-target sites {λₘ}₁^{M} in the edited and control NA collections.

5. The method of claim 1, wherein said processing includes a preliminary preprocessing of the sequencing data for adjusting said reads of said multiplexed amplifications' products/amplicons from each of the edited and control collections by carrying out at least one of the following:
trimming of sequencing adapters from said reads,
merging pair-end reads, and
filtering out low-quality reads.

6. The method of any claim 3, adapted for determining at least one indel type T of said one or more of types of adverse effect of the NA editing procedure, which belong to the Category 1 of adverse effects that is associated with INDEL activity of said NA editing procedure, and which belong to at least one class of adverse effects associated with a respective site/locus of interest λₘ₁.

7. The method of claim 6, wherein said processing of said sequencing data includes matching reads of said sequencing data to the site/locus of interest λₘ₁ by carrying out the following:
a. providing reference data indicative of at least one reference NA sequence of the at least one respective site/locus of interest λₘ₁ for which INDEL activity of said NA editing procedure is to be assessed;
b. utilizing a ‘collective’ count match condition of said template statistical model, to identify matched reads of the multiplexed amplifications products/amplicons that match the reference NA sequence of the site/locus of interest λₘ₁ in the reference data; and
thereby obtaining for the site of interest, λₘ₁, respective collections L_{Tx}(λₘ₁) and L_{M} (λₘ₁) of matched reads, in the sequencing data of the edited and control collections respectively.

8. The method of claim 7, wherein the ‘collective’ count match condition of the template statistical model of Category 1 of adverse effects is satisfied for a read to be match in case the prefix and suffix and regions of the read match prefix PRS⁺ₘ and suffix PRS⁻ₘ primer sequences (PRS⁺ₘ₁, PRS⁻ₘ₁) of the respective site of interest λₘ₁.

9. The method of claim 7, wherein sizes of said respective collections L_{Tx}(λₘ₁) and L_{M}(λₘ₁) present said ‘collective’ counts N_{Tx} and N_{Mc} of reads of amplicons which are associated with said at least one class of adverse effects involving the site λₘ₁ observed in the sequencing data of the corresponding edited and control collections.

10. The method of claim 7, wherein said processing of said sequencing data by includes segregating the collections L_{Tx}(λₘ₁) and L_{M}(λₘ₁) of reads matching the site of interest λₘ₁, to form at two sub-collections L_{Tx}(λₘ₁,T) and L_{M}(λₘ₁,T) of reads presenting a certain type T of indel observed in the matched reads from the sequencing data of the edited and control collections respectively;
wherein each indel type T is characterized by at least one of:
a size/length τ of bases introduced-to or deleted-from the matched read relative to the reference NA sequence of the site/locus of interest λₘ₁, and
a position i along the site of interest λₘ₁ at which said bases are introduced-to or deleted-from; and
wherein said segregating includes carrying out the following:
(a) aligning the matched reads in the collections L_{Tx}(λₘ₁) and L_{M}(λₘ₁) to the reference NA sequence of the site of interest λₘ₁, and
(b) identifying gaps in the aligned matched reads whereby each gap representing an indel and at least one of a position i and a length τ of the gap represents a type T of said indel; and
(c) respectively aggregating the aligned matched reads of the collections L_{Tx}(λₘ₁) and L_{M}(λₘ₁), to form the corresponding sub-collections L_{Tx}(λₘ₁,T) and L_{M}(λₘ₁,T) of reads, respectively presenting observations of said certain type T of indel, in the aligned matched reads of the sequencing data of the corresponding edited and control collections, whereby said aggregating comprises matching the identified gaps in the aligned matched reads with properties of a gap representing said certain type T of indel based on an ‘affected’ count match condition of the template statistical model of Category 1 of adverse effects, whereby said ‘affected’ count match condition of the template statistical model of Category 1 is satisfied upon fulfillment of a predetermined set one or more of the following conditions:
i) the position i of an identified gap in an aligned matched read is similar to a position î of the gap in said type T of indel;
ii) a size τ of the gap of an identified gap in an aligned matched read is similar to a size \hat{ }τ of the gap in said type T of indel;
iii) a nucleotide base sequence in the gap of an identified gap in an aligned matched read has a degree of similarity with a nucleotide base sequence of said type T of indel above a certain threshold.

11. The method of claim 10, wherein said indel type T is characterized by both said size/length τ of bases and said position i.

12. The method of claim 10, wherein sizes of said respective sub-collections L_{Tx}(λₘ₁,T) and L_{M}(λₘ₁,T) present said ‘affected’ counts n_{Tx} and n_{Mc} of reads of amplicons, in which said particular type T of adverse effect is observed.

13. The method of claim 6, wherein the template statistical model provided for the INDEL activity of said NA editing procedure comprises a statistical classifier comprising a Maximum A Posteriori (MAP) estimator.

14-32. (canceled)

33. A method for determining effects of Nucleic Acid (NA) editing procedure, the method comprising:
receiving sequencing data resulting from sequencing of multiplexed amplifications products/amplicons of a first and second collections of NA sequences, such that the sequencing data is indicative of pluralities of reads, R_{Tx}={rᵢ^{Tx}} and R_{Mc}={rⱼ^{Mc}}, of the multiplexed amplifications' products/amplicons from each of the first and second collections, whereby:
said first and second collections of NA sequences originate from the same NA source, such that the first collections of NA sequences is an edited collection of NA sequences from said NA source to which a certain NA editing procedure is applied, and the second collection is a control (mock) collection of NA sequences of said NA source to which said NA editing procedure is not applied;
the multiplexed amplifications of the edited and control collections are conducted with similar set of a plurality of primer molecule types {PRₜ};
said set of a plurality of primer molecule types {PRₜ} is designed to provide amplification of expected editing sites {λₘ₁}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites;
processing said sequencing data by a processor for constructing, per each particular type of adverse effect of one or more types of possible adverse effects of said NA editing procedure, a statistical model of occurrence of said type of adverse effect by the NA editing procedure, and applying said statistical model to said sequencing data to statistically determine actual occurrence of said type of adverse effect by the NA editing procedure; and
outputting data indicative of whether said each type of adverse effect by actually occurs due to the NA editing procedure, to thereby enable determination of adverse effects of the NA editing procedure,
wherein said one or more types of adverse effect are classified to one or more classes of adverse effects, each class being characterized by the one or two involving sites [λₘ₁,λₘ₂], with which adverse effects of the class are involved, whereby each class belongs to one of two categories of adverse effects:
Category 1 (INDELs): adverse effects involving one site [λₘ₁₌ₘ₂] where m1=m2; and
Category 2 (TRANSLOCATIONs): adverse effects involving two sites [λₘ₁,λₘ₂] where m1≠m2; and
wherein said processing said sequencing data for the particular type of adverse effect comprises carrying out the following operations a. to d. for at least one of said two categories of adverse effects:
a. providing a template statistical model corresponding to the category of said particular type of adverse effect;
b. processing the sequencing data according to the class [λₘ₁,λₘ₂] of said particular type of adverse effect to determine or assess respective ‘collective’ counts N_{Tx} and N_{Mc} of reads of amplicons which are associated with the involving sites [λₘ₁,λₘ₂] of said class in the sequencing data of each of the edited and control collections;
c. processing the sequencing data according to the particular type of adverse effect to determine or assess respective ‘affected’ counts n_{Tx} and n_{Mc} of reads of amplicons in which said particular type of adverse effect is observed;
d. applying said template statistical model to the respective ‘collective’ counts N_{Tx} and N_{Mc} of reads of amplicons which are associated with the involving sites [λₘ₁,λₘ₂] in the edited and control collections and to the respective ‘affected’ counts n_{Tx} and n_{Mc} of reads of amplicons in which said particular type of adverse effect is observed in the reads of amplicons which are associated with the edited and control collections; and
thereby statistically determining whether said particular type of adverse effect is affected by the NA editing procedure.

34. A method for determining translocation adverse effects of Nucleic Acid (NA) editing procedure, the method comprising:
receiving sequencing data resulting from sequencing of multiplexed amplifications products/amplicons of a first and second collections of NA sequences, such that the sequencing data is indicative of pluralities of reads, R_{Tx}={rᵢ^{Tx}} and R_{Mc}={rⱼ^{Mc}}, of the multiplexed amplifications' products/amplicons from each of the first and second collections, whereby:
said first and second collections of NA sequences originate from the same NA source, such that the first collections of NA sequences is an edited collection of NA sequences from said NA source to which a certain NA editing procedure is applied, and the second collection is a control (mock) collection of NA sequences of said NA source to which said NA editing procedure is not applied;
the multiplexed amplifications of the edited and control collections are conducted with similar set of a plurality of primer molecule types {PRₜ};
said set of a plurality of primer molecule types {PRₜ} is designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites; and said set of the plurality of primer molecule types comprises, or constituted by, match pairs (PRM⁺ₘ, PRM⁻ₘ) of forward PRM⁺ₘ and revers PRM⁻ₘ primer molecule types {(PRM⁺ₘ, PRM⁻ₘ)}₁^{M}∈{PRₜ} suitable for amplification of said on-target and off-target sites {λₘ}₁^{M} in the edited and control NA collections;
processing the sequencing data to identify at least one type or species of translocation adverse effect involving two different sites [λₘ₁,λₘ₂] of the expected on-target and off-target sites of said NA editing procedure, whereby said processing comprises:
counting reads rᵢ of pluralities of said reads R_{Tx}={rᵢ^{Tx}} and R_{Mc} {rⱼ^{Mc}} of the edited and control collections which satisfy a single site partial match condition with respect to at least one of the two different sites [λₘ₁,λₘ₂] and thereby assessing respective ‘collective’ counts N_{Tx} and N_{Mc} of reads of the edit and control collection in which at least one of the two different sites [λₘ₁,λₘ₂] is involved;
counting reads rᵢ of the pluralities of said reads R_{Tx}={rᵢ^{Tx}} and R_{Mc}={rⱼ^{Mc}} of the edited and control collections which satisfy a double site match condition DS associated with said type or species of the translocation adverse effect, to determine respective ‘affected’ counts n_{Tx} and n_{Mc} of reads of the edit and control collection satisfying said double site partial match condition DS; and
statistically determining whether the ‘affected’ count n_{Tx} in the edit collection is observed due to said at least one type or species of translocation adverse effect occurring in the NA editing procedure, by applying a selected statistical distribution model to said ‘collective’ and ‘affected’ counts N_{Tx}, N_{Mc}, n_{Tx} and n_{Mc}.

35-40. (canceled)

41. A system for determining and outputting data indicative of the effects of Nucleic Acid (NA) editing procedure, comprising:
an input adapted to receive sequencing data indicative of respective read results comprising of pluralities of reads, R_{Tx}={rᵢ^{Tx}} and R_{Mc}={rⱼ^{Mc}} obtained by sequencing of multiplexed amplifications products/amplicons of first and second collections of NA sequences, whereby the first collection is an edited collection of NA sequences to which a certain NA editing procedure was applied, and the second collection is a control collection of NA sequences to which said NA editing procedure was not applied;
a memory or a section thereof, for storing the respective pluralities of reads, R_{Tx}={rᵢ^{Tx}} and R_{Mc}={rⱼ^{Mc}}, of the multiplexed amplifications' products/amplicons from each of the first and second collections;
a memory or a section thereof, for storing data indicative of the set of the plurality of primer molecule types {PRₜ} used for said multiplexed amplifications of expected editing sites {λₘ}₁^{M} of the NA editing procedure;
a memory or a section thereof, for storing reference data indicative of at least one reference NA sequence of at least one respective site λₘ of the expected editing sites {λₘ}₁^{M} of the NA editing procedure;
a memory or a section thereof, for storing at least one template statistical model corresponding to at least one category of adverse effects; and
a processor for processing said sequencing data based on said reference data and the template statistical model for applying said template statistical model to said sequencing data to statistically determine occurrence of at least one type of adverse effect of said at least one category, due to the NA editing procedure; and
an output for outputting data indicative of the statistically determined occurrence of at least one type of adverse effect by the NA editing procedure.

42. The system of claim 41, wherein said processor is adapted to process said sequencing data for said type of adverse effect by carrying out the following:
a. retrieving from the reference data stored in said memory or section thereof, reference NA sequences of the one or two sites [λₘ₁,λₘ₂] participating in said type of adverse effect;
b. obtaining from said memory or section thereof, a template statistical model corresponding to said type of adverse effect;
c. constructing a statistical model for said type of adverse effect based on said template statistical model and the reference data, by carrying out the following:
processing said sequencing data of the first and second collection to determine or assess respective ‘collective’ counts N_{Tx} and N_{Mc} of reads of amplicons which are associated with the one or two sites [λₘ₁,λₘ₂] participating in said particular type of adverse effect, by matching said amplicons to the reference NA sequences of said one or two sites [λₘ₁,λₘ₂] according to a ‘collective’ count match condition designated by said template statistical model, and respectively counting the reads of amplicons of said first and second collections, which satisfy said ‘collective’ count match condition, to thereby determine or assess the respective ‘collective’ counts N_{Tx} and N_{Mc};
processing said sequencing data of the first and second collection to determine or assess respective ‘affected’ counts n_{Tx} and n_{Mc} of reads of affected amplicons in which said particular type of adverse effect is observed, by matching said amplicons to the reference NA sequences of said one or two sites [λₘ₁,λₘ₂] according to an ‘affected’ count match condition designated by said template statistical model, and respectively counting the reads of amplicons of said first and second collections, which satisfy said ‘affected’ count match condition, to thereby determine or assess the respective ‘affected’ counts n_{Tx} and n_{Mc};
d. statistically determining whether said particular type of adverse effect occurs due to the NA editing procedure, by applying said statistical model to the sequencing data, whereby said applying comprises:
utilizing a statistical classifier of the template statistical model; and
classifying the occurrence of said particular type of adverse effect according to said statistical classifier based on said ‘collective’ counts N_{Tx} and N_{Mc} and said ‘affected’ counts n_{Tx} and n_{Mc}.

43. The system of claim 41, configured and operable for determining said probability of occurrence for adverse effects of one or both of the following categories:
Category 1 (INDELs): adverse effects involving one site [λₘ₁₌λₘ₂] where m1=m2; and
Category 2 (TRANSLOCATIONs): adverse effects involving two sites [λₘ₁,λₘ₂] where m1≠m2.

44. The system of claim 41, comprising a sequencing utility capable of said sequencing of the multiplexed amplifications products/amplicons of the first and second collections of NA sequences; and wherein said input is connectable to the sequencing utility for receiving said sequencing data therefrom.

45. A kit for determining effects of a NA editing procedure, the kit comprising:
a set of a plurality of primer molecule types {PRₜ} designed to provide amplification of expected editing sites {λₘ}₁^{M} of the NA editing procedure, whereby the expected editing sites {λₘ}₁^{M} include at least one on-target site {λ₁} and one or more off-target sites {λₘ}₂^{M}, where λₘ represents an off-target or on-target site indexed m, and M is a number of the expected on-target and off-target sites; and
a system according to claim 41.

46. The kit of claim 45, wherein said set of the plurality of primer molecule types {PRₜ} comprises matched pairs (PRM⁺ₘ, PRM⁻ₘ) of forward PRM⁺ₘ and reverse PRM⁻ₘ primer molecule types {(PRM⁺ₘ, PRM⁻ₘ)}₁^{M}∈{PRₜ} suitable for amplification of said on-target and off-target sites {λₘ}₁^{M}.

47. (canceled)

48. A kit for determining effects of a NA editing procedure, the kit comprising a set of a plurality of primer molecule types {PRₜ} designed to provide amplification of expected editing sites {λₘ} of the NA editing procedure, whereby the expected editing sites {λₘ} include at least one on-target site λ₁ and one or more off-target sites {λₘ}2, where λₘ represents an off-target or on-target site indexed m; and wherein said set of the plurality of primer molecule types {PRₜ} comprises pairs (PRM⁺, PRM⁻) of forward PRM⁺ and reverse PRM⁻ primer molecule types (PRM⁺, PRM⁻)∈{PRₜ} suitable for amplification of said on-target and off-target sites {λₘ} such that each respective forward and revers primer molecule, PRM⁺ and PRM⁻, include at least one of a forward and revers adapters;
characterized in that said plurality of primer types {PRₜ} includes:
forward primer molecules PRM^{+A+} including forward adapters;
forward primer molecules PRM^{+A-} including revers adapters;
reverse primer molecules PRM^{-A+} including forward adapters;
reverse primer molecules PRM^{-A-} including revers adapters;
thereby enabling sequencing of all possible translocation species between at least one pair of the editing sites {λₘ}₁^{M}.

49-53. (canceled)
